(19) United States
(12) Patent Application Publication
(10) Pub. No.: US 2012/0022355 A1
(43) Pub. Date: Jan. 26, 2012

(54) Integrated electrophysiology and ultrasound imaging system

(76) Inventors: Charles Bryan Byrd, Oakdale, MN (US); Praveen Dala-Krishna, Sicklerville, NJ (US); David A. Jenkins, Sebastian, FL (US)

(21) Appl. No.: 13/187,192

(22) Filed: Jul. 20, 2011

Related U.S. Application Data
(62) Division of application No. 11/772,167, filed on Jun. 30, 2007, now abandoned.
Application No. 11/772,167 is a continuation-in-part of application No. 11/610,778, filed on Dec. 14, 2006, now abandoned.
Application No. 11/610,778 is a continuation-in-part of application No. 10/997,898, filed on Nov. 29, 2004, now abandoned.
Application No. 10/997,898 is a continuation-in-part of application No. 10/345,806, filed on Jan. 16, 2003, now Pat. No. 6,908,434.
Application No. 10/345,806 is a continuation-in-part of application No. 10/998,039, filed on Nov. 29, 2004, now Pat. No. 7,648,462.
(60) Provisional application No. 60/349,060, filed on Jan. 16, 2002.

(51) Int. Cl. A61B 5/04; A61B 8/12

(52) U.S. Cl. 600/373

(57) ABSTRACT

An integrated electrophysiology and ultrasound imaging system includes a workstation, electrophysiology processing circuits, a compact ultrasound imaging system having a combination of an isolation circuit, an ultrasound signal generator, and a beam former within a single unit. The integrated workstation provides a single control interface and data display for the electrophysiology and ultrasound imaging subsystems. Integrating the control of electrophysiology and ultrasound imaging equipment within a single workstation reduces clinician workload.

### CROSS-REFERENCE TO RELATED APPLICATIONS

The present application is a divisional of U.S application Ser. No. 11/772,167, filed 30 Jun. 2007, now pending (the '167 application), which is a continuation-in-part of U.S. patent application Ser. No. 11/610,778, filed 14 Dec. 2006, now pending (the '778 application). The present application is also a continuation-in-part of U.S. application Ser. No. 10/997,898, filed 29 Nov. 2004, now abandoned (the '898 application), which is a continuation-in-part of U.S. patent application Ser. No. 10/345,806, filed 16 Jan. 2003, now U.S. Pat. No. 6,908,434 (the '806 application), which claims the benefit of U.S. provisional application No. 60/349,060, filed 16 Jan. 2002 (the '060 application). The present application is also a continuation-in-part of U.S. application Ser. No. 10/998,039, filed 29 Nov. 2004, now U.S. Pat. No. 7,648,462 (the '039 application), which is a continuation-in-part of the '806 application, which claims the benefit of the '060 application. The '167 application, the '778 application, the '898 application, the '806 application, the '060 application, and the '039 application are all hereby incorporated by reference in their entirety as though fully set forth herein.

### FIELD OF THE INVENTION

The present invention relates to medical diagnostic systems, and more particularly to an integrated electrophysiology and ultrasound imaging catheter system.

### BACKGROUND OF THE INVENTION

Advancements in miniaturization of ultrasound technology has enabled the commercialization of catheters including phased array ultrasound imaging transducers small enough to be positioned within a patient's body via intravenous cannulation. By imaging vessels and organs, including the heart, from the inside, such miniature ultrasound transducers have enabled physicians to obtain diagnostic images available by no other means.

Another important cardiac diagnostic technology is intracardiac electrophysiology recorders, which include catheters with multiple electrodes on a distal end that can be positioned within a patient's heart to record electrical signals passing through heart tissue. Electrophysiology catheters and the associated analyzer equipment provide the clinician with important information regarding root causes of heart arrhythmia, dyssynchrony and other irregular heart beat maladies.

Given the diagnostic advantages of intracardiac ultrasound imaging and electrophysiology analysis, many diagnostic procedures make use of both technologies to better diagnose heart disease. However, the large cart used to hold ultrasound imaging equipment and the large cart used to hold electrophysiology equipment take valuable up space in the catheterization lab. Just as important, the separate ultrasound imaging and electrophysiology systems require the operating clinicians' attention, increasing their workload during the risky catheterization procedure.

### SUMMARY OF THE INVENTION

The present invention is directed toward providing an integrated electrophysiology and ultrasound imaging system which can provide a single operating interface for these diverse medical diagnostic systems. A single workstation is able to receive and analyze intracardiac electrophysiology signals, such as from intracardiac electrophysiology catheters while controlling and receiving data from an ultrasound imaging catheter system. In an embodiment, a compact, portable ultrasound beamformer unit is connected to and controlled by a workstation that is also configured to perform/control electrophysiology analysis. The portable ultrasound beamformer unit includes a compact, integrated ultrasound pulse generation, beam forming, and electrical isolation unit with connectors for connecting to one or more ultrasound transducer arrays and an image display unit. An ultrasound imaging catheter can may be connected to the ultrasound beamformer unit.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention.

FIG. 1A-1E are block diagrams of alternative embodiments of the present invention.

FIG. 2A and 2B are illustrations of an intra-cardiac ultrasound imaging catheter and an electrophysiology catheter located in and near the right ventricular cavity.

FIG. 3 is a diagram of a catheter transducer array with temperature sensor.

FIG. 4 is an illustration of an electrophysiology catheter.

FIG. 5 is a schematic of the isolation and temperature monitoring circuit according to an embodiment.

FIG. 6 is a block diagram of an embodiment of the integrated system.

FIG. 7 is a block diagram of an image processing computer of an embodiment.

FIG. 8 is a sample display of an ultrasound image from a cardiac ultrasound transducer.

FIG. 9 is block diagram of another embodiment.

FIG. 10 is an illustration of an example connector for an embodiment.

FIG. 11 is an illustration of an integrated electrophysiology and ultrasound imaging catheter system according to an embodiment.

FIG. 12 is an illustration of an integrated electrophysiology and ultrasound imaging catheter system according to another embodiment.

FIG. 13 is an example integrated display of electrophysiology data and an ultrasound image according to an embodiment.

FIG. 14 is an example integrated display of electrophysiology data, an ultrasound image and a fluoroscopy image according to an embodiment.

FIG. 15 is a flow block diagram of an embodiment method of using the system embodiment illustrated in FIG. 11.

### DETAILED DESCRIPTION OF THE PREFERRED EMBODIMENTS

Various embodiments of the present invention will be described in detail with reference to the accompanying drawings. Wherever possible, the same reference numbers will be used throughout the drawings to refer to the same or like parts.

As used herein, the terms “about” or “approximately” for any numerical values or ranges indicate suitable dimensional tolerances that allow the part or collection of components to function for their intended purposes as described herein. Also, as used herein, the terms “patient”, “host”, and “subject” refer to any human or animal subject and are not intended to limit the systems or methods to human use. Further, embodiments of the invention will be described for use with an intracardiac ultrasound transducer array catheter. However, the embodiments may be applicable to any medical ultrasound transducer.

Common cardiac diagnostic procedures involve positioning electrophysiology catheters near or within a patient's heart in order to obtain and record electrical signals passing through the heart with each heartbeat to assess the heart's health. Frequently an ultrasound imaging catheter will also be positioned with the patient's heart to obtain images of portions of the heart, measure blood flow through the heart chambers and valves and observe the movement of the heart with each heartbeat. These procedures are typically conducted simultaneously in a hospital “cath lab” since they both involve catheterization requiring positioning of catheter tips at precise locations in the heart. Also, cardiologist will consider electrophysiology data in conjunction with cardiac ultrasound data when diagnosing several types of cardiac disease.

Heretofore, the electrophysiology workstation and ultrasound imaging workstation have been separate systems housed in different equipment, physically and electrically isolated one from another. The functions and designs of electrophysiology and ultrasound systems are incompatible which, until this invention, have required the separate equipment. In particular, the electrophysiology workstation must sense and record extremely weak voltages that pass through heart tissue and picked up by small electrodes on the electrophysiology electrodes. The electrophysiology workstation must amplify these very weak signals without distortion while filtering out external electrical noise. In contrast, the ultrasound imaging workstation must generate high frequency electrical pulses for each of the ultrasound transducers (typically 64 transducers) within the ultrasound imaging catheter, provide these pulses to the ultrasound imaging catheter, and then receive the high frequency echo signals returning from the transducers. The generation and processing of ultrasound electrical pulses would be a source of electrical noise that would interfere with electrophysiology readings if the two systems were integrated within a single workstation. Solutions to such interference might be possible, but only with expensive and complex electrical and physical isolation measures that would increase the cost of an integrated system beyond the cost of separate systems. Thus, the conflicting design requirements and electrical functions of electrophysiology and ultrasound systems have kept the two systems separated.

As a consequence, clinicians conducting electrophysiology and intracardiac ultrasound imaging examinations must monitor and control two separate systems on separate workstations. This adds to the clinician's workload. Also, the presence of two separate systems take up space in the cath lab which is already crowded with catheterization equipment, fluoroscopy systems and other diagnostic equipment.

The present invention integrates the control and display function for an intracardiac ultrasound imaging system within an electrophysiology analyzer by employing an integrated beamformer and isolation box to provide electrical isolation of ultrasound generation/processing electronics from the electronic filters and amplifiers of the electrophysiology equipment. As used herein, an electrophysiology analyzer (or electrophysiology equipment) refers to a multichannel electrocardiogram (ECG) system suitable for receiving and analyzing electrical signals received by one or more intracardiac electrophysiology catheters. A suitable electrophysiology analyzer for use in the various embodiments is the EP-WorkMate® electrophysiology workstation manufactured by EP MedSystems, Inc. of West Berlin, N.J.

In overview, an embodiment provides an integrated workstation with electrophysiology analyzer processing circuitry that includes communication interfaces and functionality for receiving processed ultrasound data from and providing commands to an integrated ultrasound beamformer and isolation box. Image processing and display capabilities are included in the integrated workstation with the capability of displaying both electrophysiology and ultrasound data and images, as well as combined images. The integrated workstation includes a communication interface for sending command signals to the ultrasound system. Such signals will include the standard ultrasound system commands, but may also include ECG signals which may be used to gate or otherwise control ultrasound imaging. The integrated workstation further includes control interface displays and human interface devices (e.g., keyboard, mouse, light pen, touch screen display, etc.) to enable a user to control both electrophysiology and ultrasound imaging process from a single interface. By the eliminating the need for a separate ultrasound analyzer and display system, the various embodiments reduces the number of separate systems needed within the hospital's cath lab.

In an embodiment, a cable (or multiple cables) provide an electrical connection between the integrated electrophysiology/ultrasound workstation and the beamformer/isolation box. In another embodiment, a wireless data link provides a data communication connection between the integrated electrophysiology/ultrasound workstation and the integrated beamformer/isolation box. Embodiments using wireless data and control links reduces problems with electronic noise of ultrasound pulses leaking into electrophysiology data.

In an embodiment, the system also includes software for generating an ultrasound image display within or adjoining the electrophysiology display. The display may generate a combined ultrasound and electrophysiology display, such as overlaying EP data on ultrasound images, or side-by-side displays.

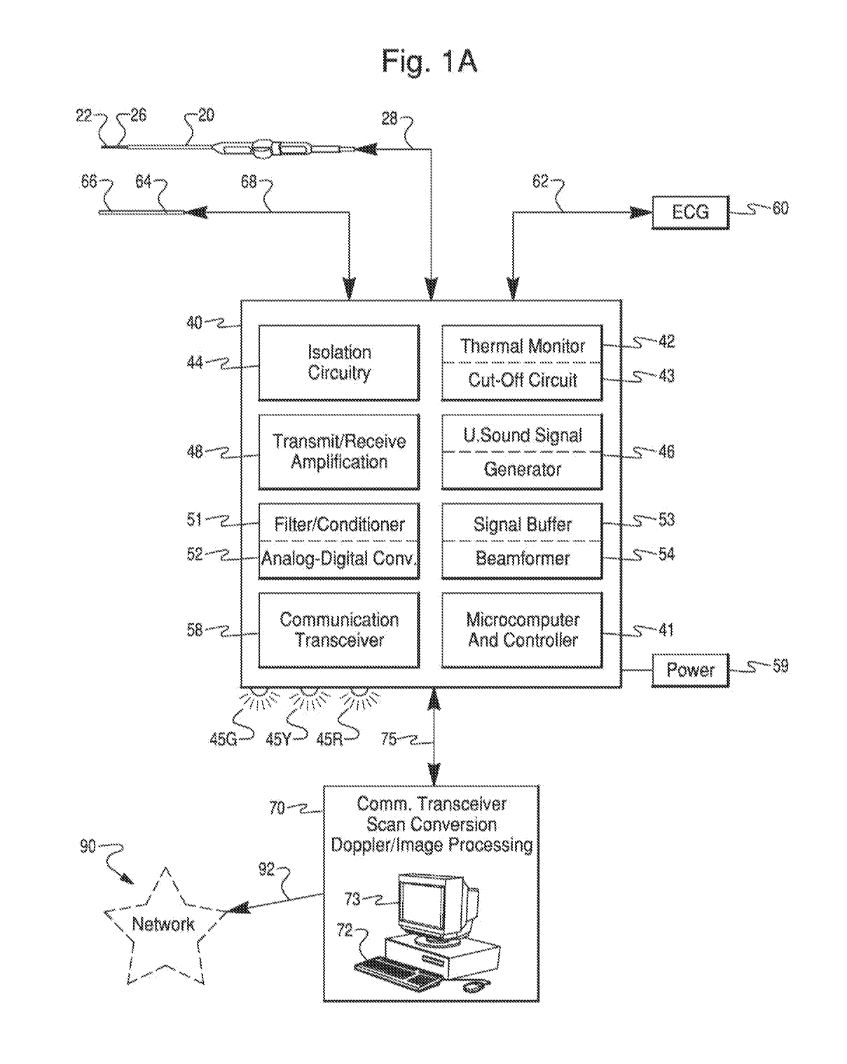

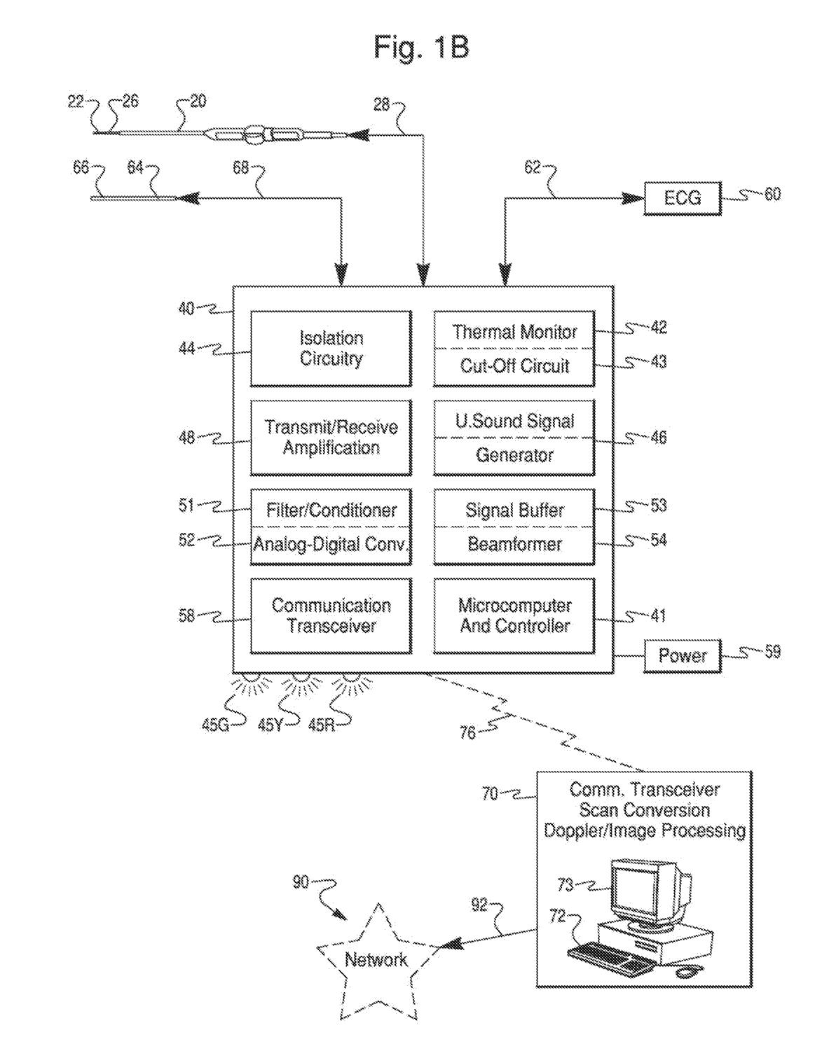

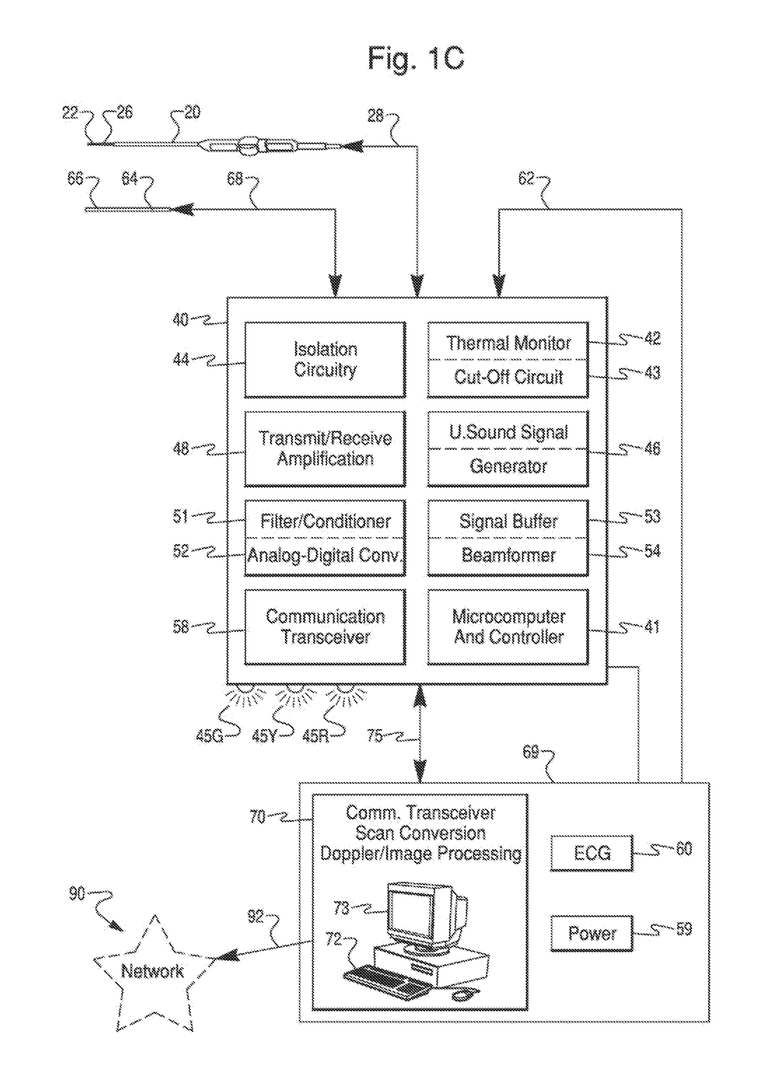

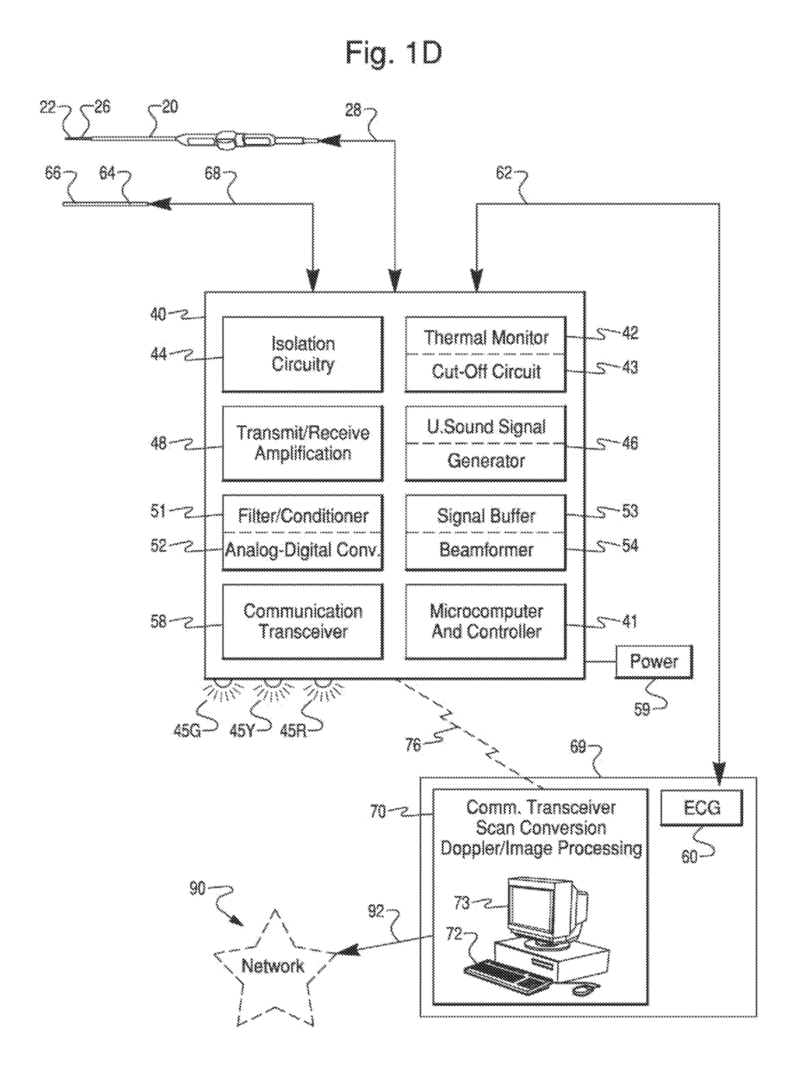

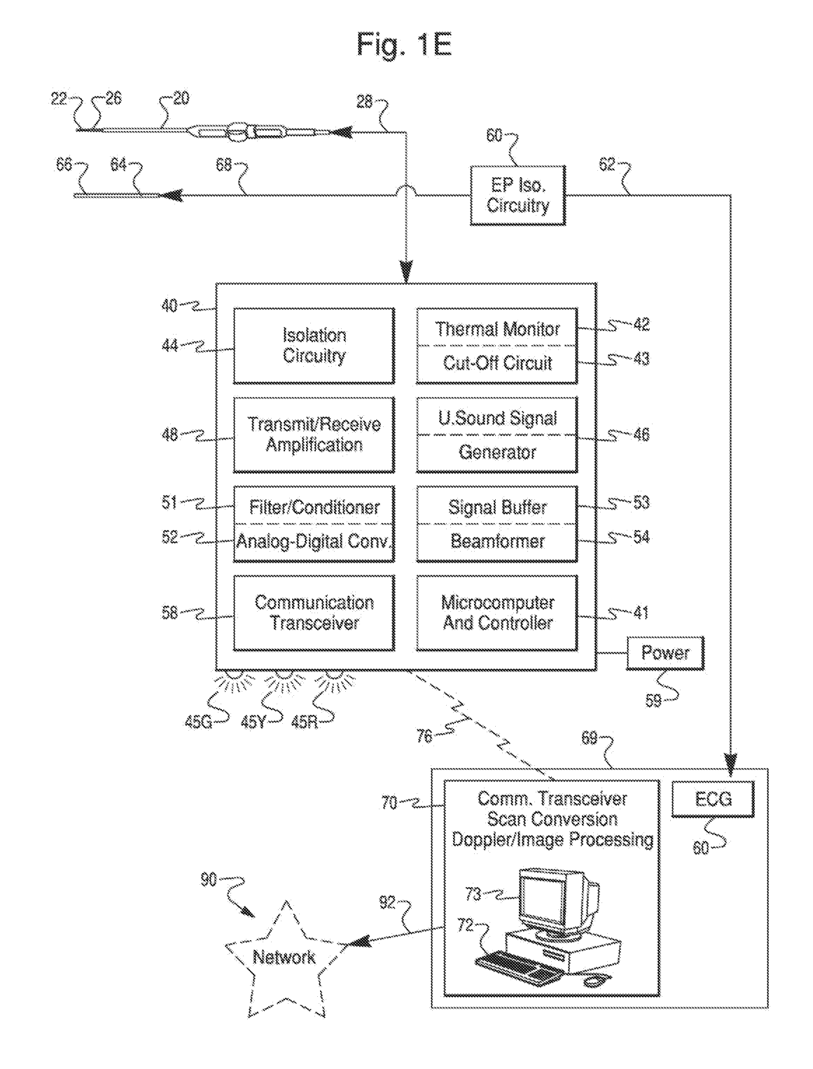

Main elements of the various embodiments are illustrated in the block diagrams of FIGS. 1A-1D. The embodiments provide an integrated electrophysiology/ultrasound imaging system that includes an ultrasound beamformer 40, an electrophysiology analyzer 60 (abbreviated “ECG” in the figures for electrocardiogram) and a display unit 70 including a display 73 and user interface devices 72. The ultrasound beamformer 40 includes an electrical interface for electrically connecting an ultrasound transducer array 22 carried by or positioned on a catheter 20 by an ultrasound signal cable 28, and one or more electrical interfaces for connecting electrophysiology catheters 64 by a EP signal cable 68. In some embodiments, the ultrasound beamformer 40, electrophysiology analyzer 60 and display unit 70 are packaged in separate units electronically connected and configured as an integrated system as illustrated in FIG. 1A and 1B. In some embodiments, the electrophysiology analyzer 60 and display unit 70 are packaged in a single workstation 69 as illustrated in FIG. 1C and 1D. The ultrasound beamformer 40 can be connected to the display unit 70 by a wired data interface 75 (shown in FIG. 1A and 1C), a wireless data interface 76 (shown in FIG. 1B and 1D), or a fiber optic data interface (not shown specifically but diagrammatically the same as FIG. 1A and 1C).

For all medical imaging technologies, patient safety is of paramount concern. For imaging technologies involving intrabody probes (e.g., ultrasound imaging catheters, electrophysiology (EP) catheters, ablation catheters, etc.), particular attention is paid to protecting the patient from unintended electrical currents and power emissions within the patient's body. For example, testing has shown that leakage currents of sufficient strength can cause muscle stimulation, which may be detrimental to the patient undergoing intrabody imaging. As such, industry approved electrical safety standards (e.g., for isolation, grounding, and leakage current) have been established for medical devices, such as national standards set by the Association for Advancement of Medical Instrumentation, limiting leakage currents from intracardiac probes to less than 50 microamperes.

In typical catheter based probes, electrical shielding or insulation is provided by way of a robust catheter body to satisfy the industry approved electrical safety standards. Shielding alone, however, may be unsatisfactory for some implementations, as substantial shielding increases the thickness of the catheter body. Induced currents may also arise from the catheters acting as an antenna picking up electromagnetic energy radiated by electronic equipment present in a typical electrophysiology lab. In some instances, the shielding may be inadvertently damaged and, thus, not provide adequate protection. Thus, methods and devices that enable intracardiac medical devices to meet or exceed the federally mandated electrical safety standards are highly desirable.

Published research has revealed that the human heart is more vulnerable to small currents when the currents are introduced within the heart itself, such as by percutaneous catheters. In Cardiovascular Collapse Caused by Electrocariographcally Silent 60 Hz Intracardiac Leakage Current, by C. Swerdlow et. al., the authors reported that leakage currents as low as 20 microamps may induce cardiovascular collapse when applied within the heart. Accordingly, percutaneous catheters might require greater electrical isolation than specified in more general standards in order to assure patient safety.

Such small leakage currents can readily arise, for example, from imperfect electrical insulation, condensation in circuits, faulty electronic components, ambient radio waves, and induction from surrounding circuits and magnetic fields. Further, safety standards require minimum (“creepage”) distances (such as 5 millimeters) between certain conductors to isolate a patient from possible high voltage discharges resulting from unlikely but possible component failures.

For these reasons, both intracardiac electrophysiology and ultrasound imaging catheters must be electrically isolated from the analyzer systems by means of electrical isolation circuitry 44. Heretofore, such electrical isolation has been provided by an isolation box containing isolation circuitry which connects to catheters (both ultrasound and EP catheters) on one side and the analyzer systems on the other.

Isolation circuitry 44 isolates unintended, potentially unsafe electrical currents and voltages from the transducer array 22 which contacts the patient. Examples of suitable isolation circuits are described in U.S. patent application Ser. No. 10/997,898 “Method And Apparatus For Isolating A Catheter Interface”, published as U.S. Patent Publication No. 2005/0124898 to Borovsky et al filed on Nov. 29, 2004, the entire contents of which are hereby incorporated by reference. An example of such safety methods and systems is embodied in the ViewMate® catheter ultrasound system from EP MedSystems, Inc. of West Berlin, N.J.

In various embodiments, the isolation circuitry 44 is integrated with ultrasound beamformer circuitry in an integrated ultrasound beamformer unit 40. For this reason, the system description begins with a description of the details of the ultrasound beamformer unit 40. While the following description address embodiments in which the ultrasound beamformer unit 40 is a separate box, in another embodiment this unit 40 is included on or within a structure housing the electrophysiology analyzer 60 and display unit 70.

The ultrasound beamformer unit 40 may include a housing or chassis with exterior connectors for connecting cables to other elements of the embodiment. The ultrasound beamformer unit 40 may contain optical and electronic circuitry implementing some or all of the elements described in the following paragraphs. The component elements and interconnecting circuitry of ultrasound beamformer unit 40 may include one or more large scale integrated circuits such as VLSI, ASIC, and FPGA chips mounted on one or more circuit boards which are coupled to the connectors.

A signal cable 28 delivers ultrasound signals from ultrasound beamformer unit 40 to each of the transducers in the array 22. Typically, the signal cable 28 will include at least one wire per transducer, and in an embodiment, includes a coaxial cable connected to each transducer in the array 22. In an alternative embodiment, the signal cable 28 includes fewer wires than transducers and a multiplexer circuit (not shown) configured to enable signals to and from the plurality of transducers over the wires. Typically, the signal cable 28 includes an electrical connection plug (e.g., a standard connector) on the proximal end. Providing a plug connector on the end of the cable 28 allows completion of the many electrical connections between the cable conductors and the ultrasound beamformer unit 40 since the connection can be accomplished by pressing the plug into a complementary connector in the housing 100 of the ultrasound beamformer unit 40.

The transducers in the array 22 convert the electrical signals from the ultrasound beamformer unit 40 into sound waves, which propagate into a portion of a patient's anatomy, such as the heart. The same transducer array 22 also receives ultrasound echoes reflected from anatomic structures and transforms the received sound into electrical signals (e.g., by means of the piezoelectric effect). These electrical signals are conducted via cable 28 back to the ultrasound beamformer unit 40.

Signal generator 46 generates electrical signals of ultrasonic frequencies to be provided to the ultrasound transducer array 22. The signal generator 46 can be configured to produce signals of particular wave forms, frequencies and amplitudes as desired for imaging tissue. The signal generator 46 may also be configured to generate signals with the necessary phase lag to enable the transducer array to generate a focused and steerable sound beam as well known in the art of imaging ultrasound phased array transducers. Alternatively, phase lag may be added by another circuit, such as a beam former circuit 54.

A transmit/receive multiplexer circuits 48 can be included to direct the signals generated by the generator 46 to isolation circuitry 44 and to separate out echo signals returned from isolation circuitry 44 from the generated signals.

A thermal monitoring circuit 42 and a cut-off circuit 43 may be included to mitigate possible risks to the patient that can result from excessive local heating by ultrasound. For example, the thermal monitoring circuit 42 may be connected to a temperature sensor (not shown), such as a thermoresistor (“thermistor”) positioned on the catheter near the transducer array 22. The thermal monitoring circuit 42 is preferably configured to determine from signals received from the temperature sensor when temperatures in the vicinity of the transducers exceed a safe threshold value and to trigger a safety action when the threshold is exceeded. The safety action may be the output of a cut-off signal to a cut-off circuit 43 which is configured to shut off the signal generation, disconnect transmit circuits from the transmission cable 28, or otherwise discontinue the transmission of ultrasound pulses to the transducer array 22 in response to a cut-off signal. Examples of suitable temperature sensors, thermal monitor circuits and cut off circuits are provided in U.S. patent application Ser. No. 10/998,039 entitled “Safety Systems And Methods For Ensuring Safe Use Of Intra-Cardiac Ultrasound Catheters” published as U.S. Patent Publication No. 2005/0124899 to Byrd et al. filed on Nov. 29, 2004, the entire contents of which are hereby incorporated by reference.

In an embodiment, the thermal monitor circuit 42 is configured to monitor temperature of the catheter as sensed by a thermistor on the catheter, and to transmit to the display unit 70 a temperature value that may be displayed on a monitor. The thermal monitor circuit 42 in such an embodiment may calculate the catheter temperature value or estimate the intracardiac tissue temperature value and transmit this value as digital data to the display unit 70. The thermal monitor circuit 42 may also be configured to transmit a warning when the measured temperature exceeds a threshold, such as a temperature that is elevated but still safely below a level at which tissue damage may occur. Such a warning would inform clinicians of a potentially hazardous condition to permit them to take actions to reduce heating, such as adjusting ultrasound power or duty cycle parameters, in order to avoid damaging tissue and automatic shutoff by the cut-off circuit 43. Such a warning may be transmitted to the display unit 70 as digital data for display on the monitor (image display unit).

In another embodiment, a display, such as colored light emitting diode (LED) indicators (45G, 45Y, 45R) on the ultrasound beamformer unit 40 are provided to indicate temperature information, in the alternative or in addition to displays on to the display unit 70. For example, in such an embodiment three LEDs may be provided, such as a green LED 45G to indicate a safe detected temperature, a yellow LED 45Y to indicate an elevated but marginally safe temperature, and a red LED 45R to indicate an unsafe or near unsafe temperature. In such an embodiment, the thermal monitor circuit 42 includes circuits configured to light the appropriate colored LED based upon the measured temperature. This configuration may be accomplished by the thermal monitor circuit 42 testing the sensed temperature against two threshold values, wherein the first threshold corresponds to elevated but still safe temperatures and the second threshold corresponds to unsafe or near unsafe temperatures. Thus, the thermal monitor circuit can be configured (e.g., with digital switches) to power (i.e., direct a voltage to) the green light emitting diode in response to the temperature input signal indicating a sensed temperature less than the first threshold, power the yellow light emitting diode in response to the temperature input signal indicating a sensed temperature greater than the first threshold but less that the second threshold, and power the red light emitting diode in response to the temperature input signal indicating a sensed temperature greater than the second threshold.

A filter and conditioner circuit 51 can be included in the ultrasound beamformer unit 40 to reject spurious signals that may be induced in or through cable 28.

An analog-to-digital converter (ADC) 52 can be included in the ultrasound beamformer unit 40 to frequently sample and convert the ultrasound signals from analog electrical levels to discrete digital numeric values.

A signal buffer 53 can be included to store at least a portion of the echo signals, which are returned from the transducer array 22 and which may be processed by other elements of the ultrasound beamformer unit 40. In an embodiment, a signal buffer 53 is included to store the echo signals as digital data in a random-access semiconductor memory (RAM).

Beam former 54 circuits may be included to process signals sent to and received from the transducer array 22 to enable phased-array ultrasound imaging. The beam former 54 may receive ultrasound signals from the signal generator 46 and introduce phase lags for each transducer element so that when the signals are applied to the transducer elements a narrow beam of sound emanates from the array. Also, the beam former 54 may receive signals from the transducer array and process the ultrasound echo signal data to calculate the amplitude and direction of the ultrasound echoes returned to the transducer array 22 from each of many specific angles and distances. The beam former 54 may also determine the frequency or Doppler frequency shift of the signal returned form each of selected angles and distances from the transducer array 22.

A communications transceiver 58 may be included to prepare ultrasound data for transmission out of ultrasound beamformer unit 40, typically in digital form. The communication transceiver 58 may also receive data and commands from outside the ultrasound beamformer unit 40 and convert such signals to a form usable by the ultrasound beamformer unit 40. Data transmission may by any high speed (e.g., gigabit per second) data link, such as Ethernet. In an embodiment, the communications transceiver 58 may also transmit electrophysiology signals to the electrophysiology analyzer 60.

In an embodiment, the communications transceiver 58 may include data encoding or compression capability, such as a microprocessor programmed with data encoding or compression software, so that the ultrasound data can be transmitted in a compressed format. By transmitting data in a compressed format, lower bandwidth communication links (e.g., cable or wireless data link) can be used to transmit data, or more data can be transmitted over a standard cable or wireless data link. Additionally, the control unit 41 or other modules may be configured to filter out data that need not be transmitted, such as signals or data pixels containing little or no data (i.e., pixels where little or no echoes were received), so such data need not be transmitted. In yet a further embodiment, the communications transceiver 58 may include temporary storage capability (e.g., random access memory) and be configured to manage the transmission of data at a maximum data rate consistent with the communication link even when data provided to the transceiver exceeds the maximum data rate. Suitable circuitry and software for encoding, compressing, buffering and filtering data and managing data transmission are well known in the communications arts.

The ultrasound beamformer unit 40 may include a control unit 41 which may be a microcontroller, a microprocessor, a microcomputer, or other controller circuitry (such as programmable firmware or a programmable gate array). The control unit 41 may be configured to coordinate the activity and functionality of the various elements included in the ultrasound beamformer unit 40.

In an embodiment associated with cardiac imaging, the ultrasound beamformer unit 40 may also include electrical connections for receiving signals from electrocardiogram (ECG) electrodes and for passing such signals on to an external electrocardiogram (ECG) unit 60 which may be connected to the ultrasound beamformer unit 40 through a communications interface 62. The communications interface 62 may be any wired or wireless interface. In an embodiment, the ECG electrodes can be an intracardiac ECG catheter 64 which includes one or more electrodes 66 near a distal end for sensing electrical activity in the heart. Electrical signals sensed by the electrodes 66 can be conveyed to the ultrasound beamformer unit 40 by means of an extension of the catheter 64 or a connecting cable 68. In various embodiments, the ECG catheter 64 is connected to the isolation circuitry 44 which isolates the patient from stray or fault voltage from the external ECG equipment 60. In an embodiment, signals sent by the ECG 60 through the interface 62 can be recorded or used to synchronize received ultrasound image data with the heartbeat of the patient. For example, a sequence of images may be associated with a sequence of ECG readings revealing the phases of the cardiac cycle, or images may be captured only at a specified phase of the cardiac cycle.

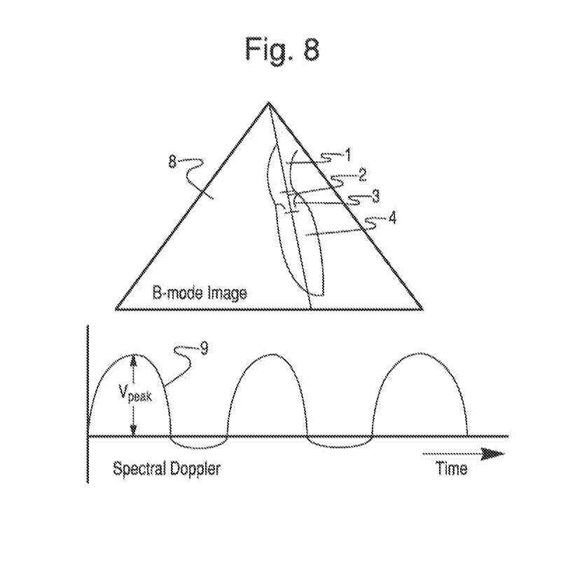

In an embodiment in which the ultrasound beamformer unit 40 may be packaged within a single housing (see FIGS. 5 and 8) or chassis, the whole unit 40 may be fabricated of components which can withstand a sterilization method. Sterilization methods include subjecting the unit 40 to gas, liquids, heat (dry or steam), radiation, or other known methods. Alternatively, or in addition, the unit 40 may be enclosed in an externally sterile enclosure, such as a plastic bag, with provision for connecting cables through the plastic bag to the unit 40. For example, the connectors may be designed so that the pins of the connector of an external electrical cable (such as cable 28) are designed to puncture the externally sterile plastic bag locally when mating with the corresponding connector of the unit 40 inside the plastic bag.

The relationship, function, and interaction of the elements which may be contained in the ultrasound beamformer unit 40 of an embodiment will be described further with reference to FIGS. 5 and 8.

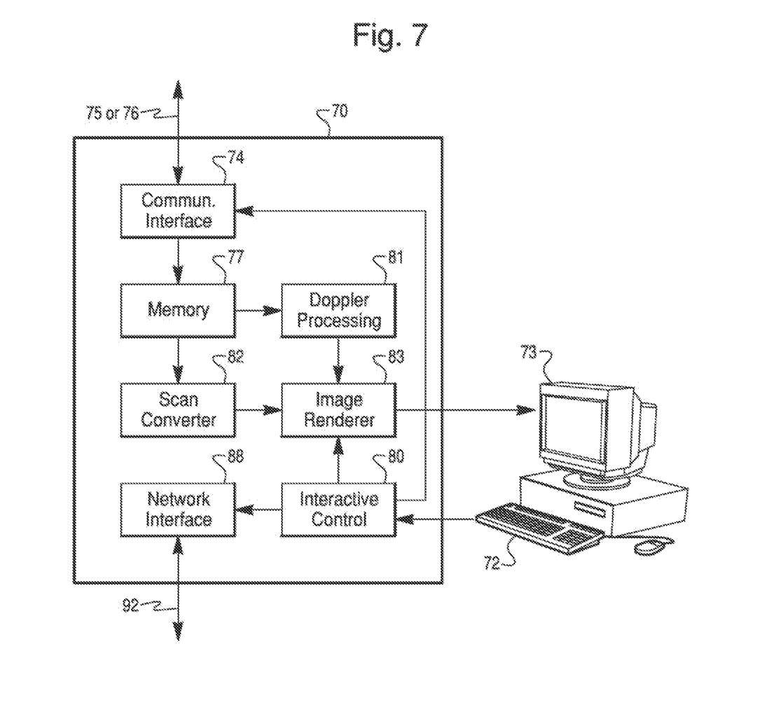

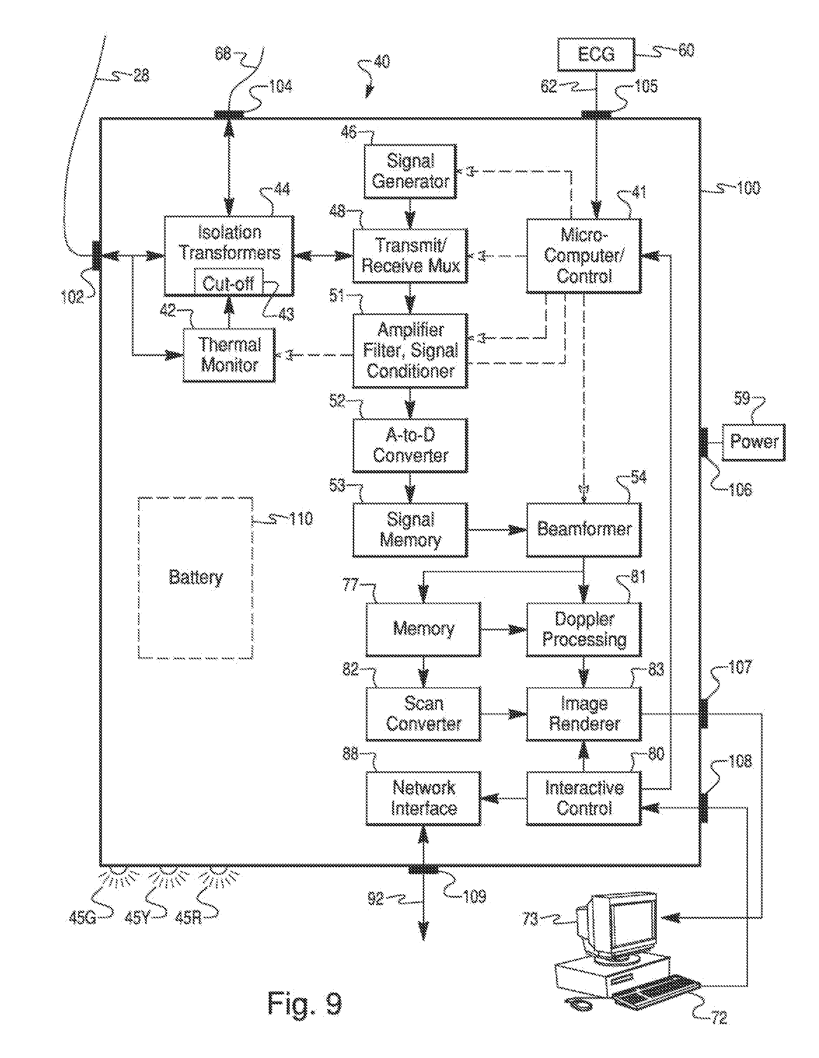

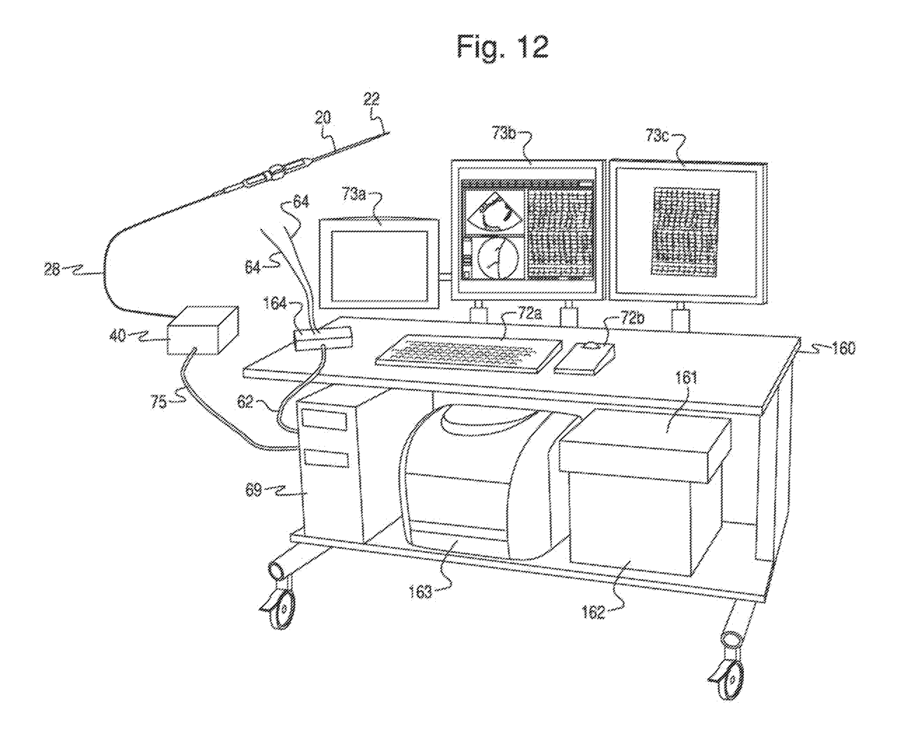

In the various embodiments, the image display unit 70 may be a computer, such as a laptop or workstation, which can be configured to perform more sophisticated image processing then is provided by the ultrasound beamformer unit 40. Such an embodiment will be described later (with respect to FIG. 7). In an embodiment (which will be discussed later in relation to FIG. 9) image display computer may include a user input device 72, and a video monitor 73. In some embodiments, the image display unit 70 includes the processor and displays of an electrophysiology analyzer workstation, a combination of the analyzer 60 and the display unit 70, such as illustrated in FIG. 12.

Optionally, there may be two (or more) separate communication interfaces 75: one interface for the ultrasound image data communicated to the display unit 70, and a second interface (not shown in FIG. 1A) for communicating configuration parameters and commands from the display unit 70 to the microcomputer 41. Additionally there may be a third separate communication interface 62 for communicating electrophysiology signals to the electrophysiology analyzer 60. These two interfaces 75 may employ the same type of communication hardware and protocol standard or two different types.

In the embodiment illustrated in FIG. 1A and 1C, pixel-based, polar-coordinate oriented ultrasonic image data can be serialized and transmitted to the image display unit 70 over the data communication interface 75. The data interface 75 may be any one or more of several standard high-speed serial or parallel data communication protocols and hardware embodiments. Example embodiments of the serial communication interface 75 include Ethernet, Universal Serial Bus (USB 2.0), FireWire (IEEE-1394), RS-232, or any other existing or future high-speed (e.g., gigabit/second) wired communication interface. As with the communications transceiver 58, the data interface 75 may include circuitry and software for encoding, compressing, buffering and filtering data, as well as managing data transmission to enable the reliable transmission of a large amount of image data through communication links of limited bandwidth. Both the ultrasound beamformer unit 40 and the image display unit 70 can contain appropriate corresponding hardware and software communication drivers, data compression and encoders, buffer memory, and data filter circuits and/or software, which are readily available commercially, such as standard off-the-shelf integrated circuits or plug-in circuit cards and well known communication algorithms.

In an alternative embodiment, the data interface 75 may be an optical cable, such as one or more fiber optic cables. This embodiment is illustrated in FIG. 1A and 1C, with the communication transceiver 58 being an optical data link transceiver. Fiber optic data links well known in the art may be used in this embodiment.

In some embodiments, the communications transceiver 58 and/or the data interface 75 are configured to recognize the particular type of communication link connected to the ultrasound beamformer unit 40, and to adapt the communication protocols, data encoding and data transmission rates to match the connected link. Such embodiments may also include software programmed in the microcomputer 41 that enables it to supervise the ultrasound beamformer unit 40 consistent with the capabilities and requirements of the connected data link. In such embodiments, the ultrasound beamformer unit 40 may include a number of different connection ports for various communication links, such as two or more of a USB port, a FireWire port, a serial data port, a parallel data port, a telephone band modem and RJ-11 port, a WiFi wireless data link and a BlueTooth wireless data link, for example. Circuitry and/or software operating in the communication modules or microprocessor 41 can be configured to sense when a particular one of the various accommodated communication links is connected, such as by sensing an electrical or radio frequency signal received by the link. Recognizing that a particular link is connected, the communications transceiver 58 and/or the data interface 75 and/or microprocessor 41 can implement the protocol, data encoding and data transmission rate that corresponds to that link. Circuits and methods for recognizing connected data links and adjusting communications protocols accordingly are well known in the digital communication arts. Such embodiments provide flexibility of use, allowing users to connect displays and processors using available or convenient cables or communication links.

Functionality within the ultrasound beamformer unit 40 can be managed and timed by a programmed microcontroller, a microprocessor, a microcomputer 41, equivalent firmware, an ASIC chip, or discrete electronic circuitry, all of which are encompassed by description references to “microcomputer” herein. The microcomputer 41 can respond to configuration parameters and commands sent from the image display unit 70 over communication interface 75. Examples of such configuration parameters and commands include the frequency of the generated ultrasound signals, the mode of operation (continuous or pulsed), depth of imaging, angular width of the active image area, amplifier gain, filter frequencies, details about the transducer array 22 (number and arrangement of transducers), and so forth.

The hardware layout and software programming needed to implement the design and programming of the ultrasound beamformer unit 40 are typical and well known to electrical and software engineers skilled in this art. Similarly, the algorithms programmed into display unit 70 are known to software engineers skilled in mathematics, computer graphics, and graphical-interface operating systems.

The image display unit 70 can perform any number of several functions. The display unit 70 can process and display the electrophysiology data provided by the electrophysiology analyzer 60 and ultrasound image data provided by the ultrasound beamformer unit 40 on a connected monitor 73 or other display, such as a large plasma screen display (not shown) coupled to the display unit 70. The display unit 70 can transmit configuration parameters and control commands to the ultrasound beamformer unit 40, where the configuration parameters and commands may be supplied by the operator of the system by means of interactive inputs from a pointing device (mouse or joystick) and keyboard attached to or part of the display unit 70. For example, the operator may inform the display unit 70 about the type of imaging catheter 20 which the display unit 70 may further translate into operational details about the transducer array 22 included in the imaging catheter 20.

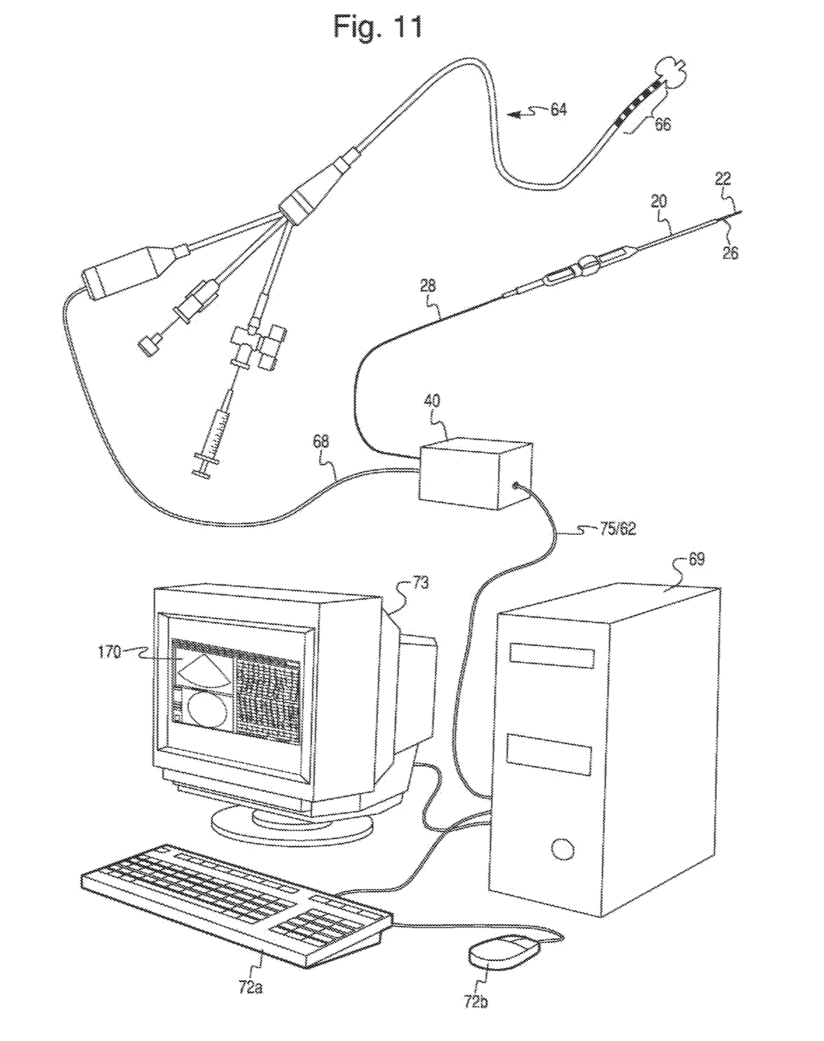

In some embodiments, a portion or all of the electrophysiology analyzer 60 functionality is integrated within the display unit 70. A typical electrophysiology system receives electrical signals from electrophysiology catheters 64, amplifies and digitizes the signals, displays the EP signals on a display 73, and stores the EP signals and electrocardiograms for off-line analysis. The electrophysiology analyzer 60 may record a large number of channels of data, such as up to 192 channels, as each electrophysiology catheter 64 may have a large number of electrode sensors 66 and multiple (e.g., two or three) electrophysiology catheter 64 may be used simultaneous. The electrophysiology system may also incorporate an integrated stimulator for providing electrical stimulation to the heart, and an interface for an ablation system for ablating heart tissue using an ablation catheter. The electrophysiology analyzer 60 may provide the clinician with a number of analysis tools and functions for viewing the EP waveforms and analyzing the data for diagnostically meaningful information. For example, the electrophysiology analyzer 60 may determine timing intervals between different ECG wave components (e.g., R-R, A-A, A-H, H-V, V-V, V-A), displayed both as waveforms and as numerical values. The analysis and display of EP data may be in real time or historical, or both such as to compare current measurements with baseline or pre-treatment measurements. Additionally, the electrophysiology analyzer 60 may provide additional functionality depending upon the processor's software, such as: activation mapping; Holter window; pace mapping tools; ablation system control and settings window; cine capture; post acquisition processing; stimulator system control window; database management with query capability; data exporting/communication capability (e.g., faxing capabilities); and other signal analysis tools. Many of these electrophysiology system functions can be performed by the processor associated with the display unit 70. Thus, as illustrated in FIGS. 1C and 1D, the electrophysiology analyzer 60 and display unit 70 may be the same physical unit, with part or all of the electrophysiology analyzer capability provided as functionality of the display unit 70 processor configured (i.e., programmed and electronically connected) with software controlled electrophysiology analysis functionality. Examples of an integrated electrophysiology analyzer 60/display unit 70 are illustrated in FIGS. 11 and 12.

Similarly, some of the functionality described herein as residing within the ultrasound beamformer unit 40 may be provided within the display unit 70 as software provided functionality of the processor. For example, in some embodiments, the image display unit 70 processor can convert the ultrasound data generated by the beam-former 54 (which may be relative to a transducer-centered polar coordinate system) into an image relative to another set of coordinates, such as a rectangular coordinate system. Such processing may not be necessary in the display unit 70, if the conversion was already preformed in the ultrasound beamformer unit 40. Techniques for converting image data from one coordinate system into another are well-known in the field of mathematics and computer graphics.

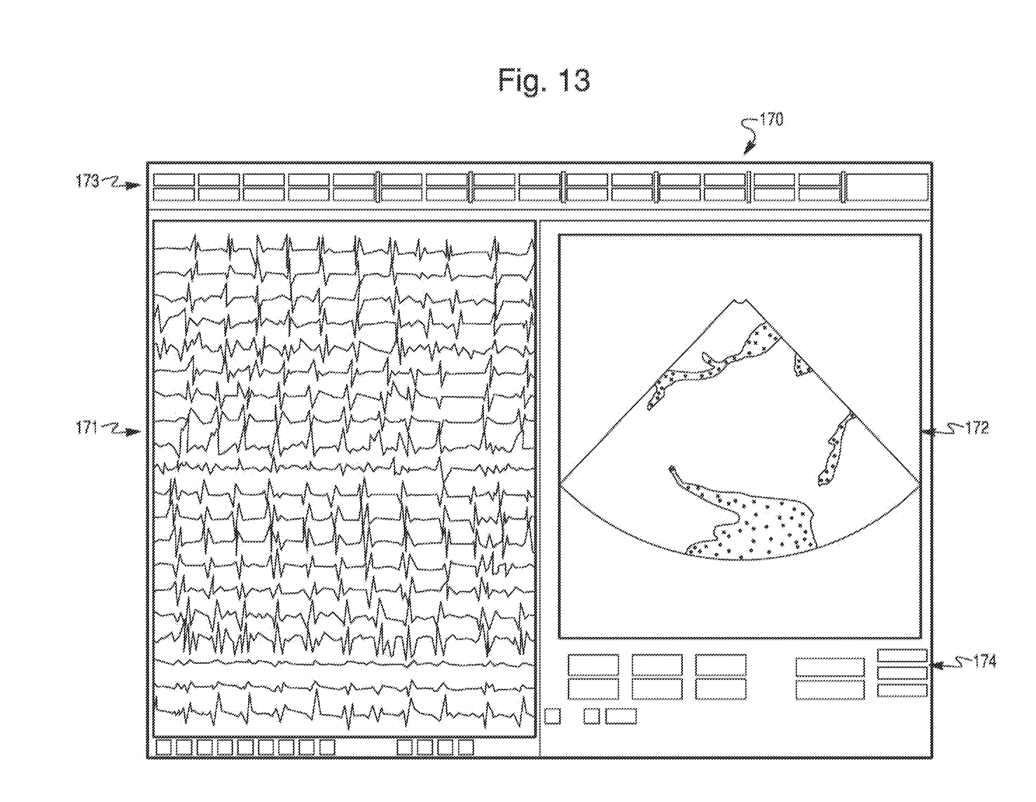

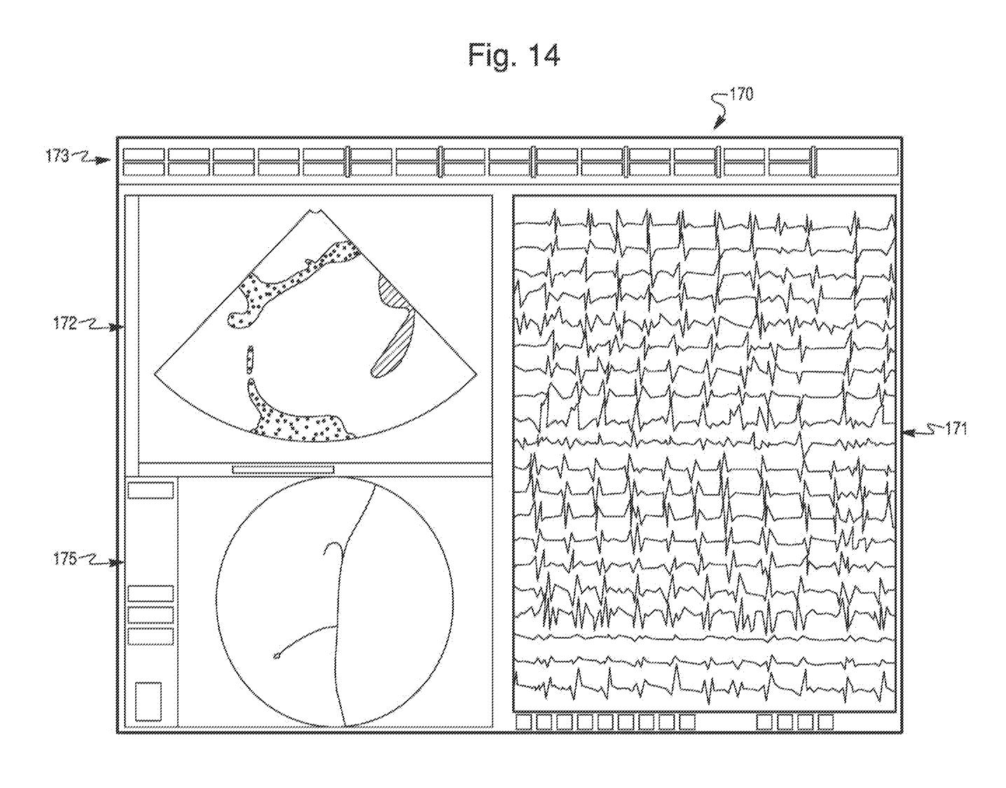

The display unit 70 may display the electrophysiology data and ultrasound image data as an image on a standard video monitor 73 or within one or more graphics windows managed by the operating system (such as Microsoft Windows XP) of the display unit 70. In addition, the display unit 70 can display textual data for the operator on the monitor 73, including, for example, information about the patient, the configuration parameter values in use by the ultrasound beamformer unit 40, and so forth. In an embodiment, the display unit 70 may provide a function that allows measuring the distance between two points on the image, as interactively selected by the operator. Additionally, the display unit 70 may provide various electrophysiology data analysis tools as described above. Examples of integrated electrophysiology and ultrasound image displays are provided in FIGS. 13 and 14 and described in more detail below.

To analyze and display an indication of motion—and specifically the velocity of movement—of locations in the image corresponding to tissue and fluid movement (i.e., blood), further Doppler frequency distribution analysis can be performed and translated into a readily understandable graphical representations. Doppler frequency analysis is well-known in the field of ultrasound medical imaging and described in more detail in the patent applications incorporated by reference herein. Fourier analysis may be used, for example, to determine the frequency distribution information and the average Doppler frequency shift for each of all points or selected points in the ultrasound image, and from which to compute the individual velocities of those points.

The display unit 70 can generate an image in which the Doppler frequency shift information communicated by the ultrasound beamformer unit 40 for each point or pixel is represented by a color hue. Since Doppler shift provides information on the speed and direction of movement of fluids and structures with respect to the transducer, various color hues can used in the display to correspond to the velocity and direction of motion. For example, red may be used to represent the maximum velocity in one direction, blue to represent the maximum velocity in the opposite direction, and colors between red and blue on the color spectrum to represent velocities in between. In another embodiment, such colors can be superimposed on the B-mode image, which is otherwise rendered as a gray-scale image wherein the brightness of each pixel depends on the amplitude of the returned ultrasound echo from the anatomical location corresponding to the pixel. Other modes of display are well known in the art, such as a plot of the distribution of Doppler frequency shifts at the points along a line in the image, and M-mode in which the movement along narrow lines is displayed.

Optionally, a connection interface 92 may connect the display unit 70 to a clinic or hospital information infrastructure 90 or the Internet. A hospital information infrastructure 90 will typically include a network of attached workstations, graphical displays, database and file servers, and the like. The optional interface 92 typically can be an Ethernet cable, a wireless WiFi interface (IEEE 802.11), or any other high-speed communications physical layer and protocol. For example, an interface 92 can allow the display unit 70 to access information from the Internet, a database, or a hospital network infrastructure 90. The display unit 70 may also transmit information outward via the interface 92, such as to store the ultrasound data on a network server or display the ultrasound images elsewhere than the display unit 70.

FIGS. 1B, 1D and 1E illustrate embodiments which electrically isolate the ultrasound beamformer unit 40 from the image display unit 70 and electrophysiology analyzer 60. The embodiments illustrated in FIG. 1B, 1D and 1E use a wireless data communication interface 76 to convey ultrasound image data to the image display unit 70. This embodiment may have safety advantages, because it electrically isolates the ultrasound beamformer unit 40 from the image display unit 70 and eliminates a data cable which can pick up and conduct stray electrical fields and electronic noise. By removing an electrical conduction path for high voltage or leakage currents, namely the wires or cable of the interface 75 between the ultrasound beamformer unit 40 and the display unit 70, this embodiment provides further patient protection from potential internal or external electrical faults and eliminates a source of electronic noise. Not only does this embodiment provide added protection from faults within display unit 70, but it can provide protection from lightening or power surges which may occur with an embodiment such as a laptop or desk-top personal computer plugged into a normal AC utility power outlet. Without sufficient isolation circuitry, lighting or surges can send a high voltage spike through the power supply of the display unit 70 and into the rest of the system through any available conductive pathway.

Physically isolating the ultrasound beamformer unit 40 from the electrophysiology analyzer 60 and display unit 70 also prevents high frequency noise from the ultrasound pulses, such as created by the ultrasound signal generator 46, from interfering with the reception, amplification and analysis of electrophysiology signals received by the electrophysiology catheter 64. As noted above, such relatively high power, high frequency pulses could overwhelm the electrophysiology signals if not properly shielded from the electrophysiology analyzer 60. A wireless data link 76, as shown in FIGS. 1B, 1D and 1E, or a fiber optic data link (as would appear as shown in FIGS. 1A and 1C) provides physical isolation of the ultrasound beamformer unit 40 from the electrophysiology analyzer 60 and display unit 70.

In the embodiment illustrated in FIG. 1E, further electrical isolation of the ultrasound beamformer unit 40 from the electrophysiology analyzer 60 is provided by using an isolation circuit 60 between the electrophysiology catheter 64 and the electrophysiology analyzer 60 that is separate from the isolation circuitry 44 between the ultrasound beamformer unit 40 and the ultrasound imaging catheter 20. In this embodiment, the isolation circuit 60 may be any of a number of commercially available isolation circuits used in electrophysiology, such as grounding the amplifier circuits (not shown separately but contained within the prior art amplifier 162 in FIG. 12) that connect to the leads from the electrophysiology catheter 64 to receive and amplify the electrophysiology signals in combination with electrically isolating (e.g., via transformer isolation) the power source from the amplifier circuits.

Commercially available wireless communications systems can be used for the wireless interface 76. For example, the wireless interface 76 may be an infrared communication interface (such as the IRDA standard), a radio-based communication interface (such as the Bluetooth, ZigBee, or 802.11 WiFi or 802.15.4 standards), or both (for example, infrared in one direction and radio in the other direction). To decrease the data processing that must be done in the ultrasound beamformer unit 40, a high speed data transmission system may be used to provide partially processed ultrasound data to the image display unit 70 for further processing. The data transmission may have a data rate of 1 megabit per second or more.

Common to the embodiments illustrated in FIGS. 1A and 1B is a power supply 59 coupled to the ultrasound beamformer unit 40. Electrical power is used both to power the processors and circuits in the ultrasound beamformer unit 40 and to provide energy for the electrical pulses which drive the transducer array 22. Power may be provided through the data cable 75 (as in the case of a USB cable embodiment), via a separate power cable connected to the display unit 70, via a separate power supply (such as a transformer connected to an AC power source), or via a self contained power source (such as a battery). For an embodiment in which the image display unit 70 does not supply power to the ultrasound beamformer unit 40, such as where the image data is communicated via a wireless data link as illustrated in FIG. 1B, the ultrasound beamformer unit 40 can be powered by a separate power source 59. Non-limiting examples of suitable separate power sources include a rechargeable battery pack, a disposable battery pack, a power supply connected to public utility power lines through an isolation transformer, a power supply engineered for safety compliance isolation from public utility power, a solar cell, a fuel cell, a charged high-capacity storage capacitor, combinations of two or more of such power sources (e.g., a rechargeable battery and a solar cell), and any other source of electrical power which may become known in the art.

A self-contained power source, such as a battery or solar cell, can provide inherent safety advantages over conventional power sources. This is because a self-contained power source 59 can be used to further isolate the patient from stray and fault currents since there need not be a power cable connected to the beam former unit. This removes the power source (such as hospital main AC power) and the power cord as sources of power spikes and fault currents. Such isolation may be further enhanced by forming the housing or chassis of the ultrasound beamformer unit 40 from non-conductive material and encasing the self-contained power source within the housing or chassis. This configuration effectively presents no return path or common ground between the power source and the patient. That is, even if there is an electrical potential difference between the ultrasound beamformer unit 40 and the patient, little current can flow between them. Further, with a self-contained power source, there is very little chance that a patient will receive high voltage from lightening or from a utility power outlet through a failed component, faulty design, or a power surge. A typical self-contained power supply 59 can supply only limited current at limited voltage, further reducing the likelihood of excessive leakage current. Also, using a self-contained power supply 59 can further reduce cables and conductors which can pick up stray electromagnetic radiation and become a source of electronic noise in the system.

In another embodiment, the power supply 59 may be power conductors parallel to or contained in the communication cable 75 of FIG. 1A, over which the image display unit 70 provides power to the ultrasound beamformer unit 40. The display unit 70 can supply power from its own supply of power such as, for example, if the interface 75 connecting the display unit 70 and the ultrasound beamformer unit 40 is embodied by a standard IEEE-1394 (Firewire) cable or a USB cable, both of which contain direct current power conductors.

If an embodiment does use an electrically conductive cable as part of the communication interface 75, then in lieu of other measures, the data communication interface 75 can include additional circuits for isolating the ultrasound beamformer unit 40 from any source of excessive leakage current or high voltage from or through the display unit 70—whether or not power is supplied over the interface 75. National and international safety organizations specify leakage current and breakdown voltage standards for various medical applications: for example, a maximum leakage current of 20 microamperes or a breakdown voltage of at least 5000 volts. By providing electrical isolation circuitry 44 within the ultrasound beamformer unit 40, greater protection for the patient can be provided against high power, power surges, and system overloads, as well as providing greater protection against or filtering of signal artifacts, signal jitter, and signal crosstalk.

Further patient electrical isolation is provided in embodiments utilizing a fiber optic data cable 75 between the ultrasound beamformer unit 40 and the display unit 70. In an alternative embodiment, further isolation may be provided by including an optical isolator module somewhere along each conductor of the interface 75. An example of an optical isolator module includes a light-emitting diode optically coupled to a photo detector and configured so that electrical signals entering the module are converted into light signals and converted back into electrical signals within the module, thereby conveying the data across an electrically isolating space. In embodiments where the data interface 75 is an optical fiber with suitable optical-electrical converters at each end of the interface 75, the optical fiber cable can be constructed to prevent or minimize electrical conduction, such as fabricating the covering from non-conducting plastics, thereby providing better electrical isolation. Optical isolation may preclude supplying electrical power through the data interface 75, so an alternative power supply 59 for the ultrasound beamformer unit 40 according to an embodiment described herein may be employed. The use of optical fiber cable or an optical isolation can also help to reduce electronic noise introduced into the ultrasound beamformer unit 40 from stray electromagnetic radiation.

In the various embodiments, the ultrasound beamformer unit 40 lies between the patient on one side and power sources and external processors/displays on the other. As such, different levels of isolation may be provided by separate isolation circuitry 44 within the ultrasound beamformer unit 40 as appropriate to particular connections. For example, by providing greater electrical isolation on connections to power and/or external processor/displays, such isolation may protect the circuitry of the ultrasound beamformer unit 40 as well as the patient from external voltage spikes and fault currents. As another example, isolation circuitry on the patient side of the ultrasound beamformer unit 40 circuitry, may reduce hardware and software complications and increase integration efficiency.

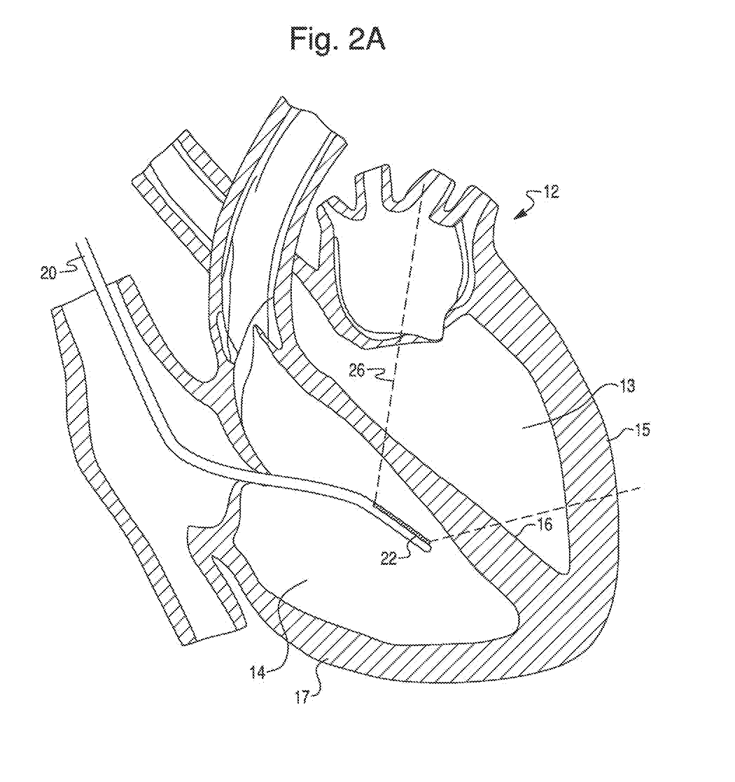

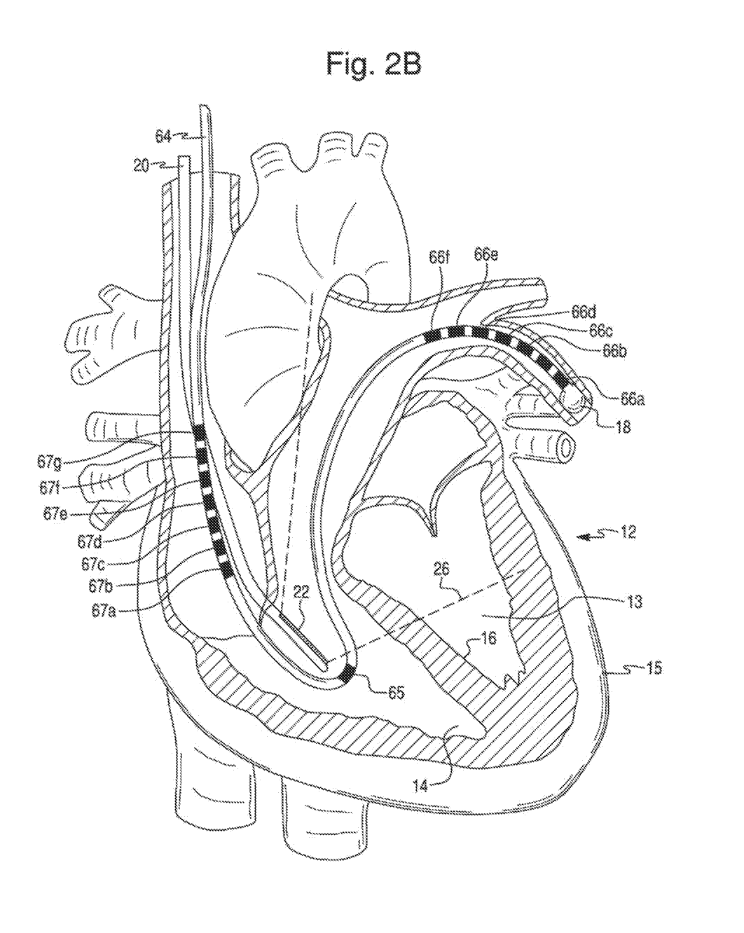

FIG. 2A depicts a simplified cross section of a human heart 12 with an ultrasonic imaging catheter 20 positioned in the right ventricle 14. The catheter 20 includes an ultrasound transducer array 22, which may image at least a portion of the heart 12. For example, the imaging view 26 afforded by the transducer array 22 may allow imaging the left ventricle 13, the ventricular walls 15, 16, 17, and other coronary structures. Usage of an embodiment may include positioning the array 22 at other locations and at other orientations within the heart (such as the right atrium), within a vein, within an artery, or within some other anatomical lumen. Insertion of the catheter 20 into a circulatory system vessel or other anatomical cavity through use of a percutaneous cannula is well known in the medical arts.

FIG. 2B depicts a cross section of a human heart 12 with an electrophysiology catheter 64 and an human heart 12 with an ultrasonic imaging catheter 20 positioned in the right ventricle 14, with the electrophysiology catheter 64 extending into the pulmonary artery. A balloon 18 may be provided on or near the distal tip to facilitate positioning of the catheter 64 within the heart 12 and the veins and arteries of the heart. The electrophysiology catheter 64 includes a plurality of electrode surfaces 67a-67g, 65 and 66a-66f positioned along its length toward the distal end. For example, as shown in

FIG. 2B, an electrophysiology catheter 64 having a series of electrodes 66a-66f near the distal end can record electrical signals passing over the upper left portion of the heart, thereby sensing right atrial electrophysiology signals, right ventricle output, certain left atrial electrophysiology signals, and temperature measurements for thermal dilution analysis, as well as provide stimulation for intracardiac defibrillation. At the same time electrodes 67a-67g positioned a distance removed from the distal end can sense electrical signals passing through the upper right portion of the heart, thereby sensing signals in the right atrium and the SA node. Reference electrodes 65 on the catheter 64 can sense the reference electrical condition of the heart, so that the electrophysiology equipment can compare the voltages received at electrodes 67a-67g and 66a-66f to a reference voltage measured within the heart.

Simultaneously positioning an ultrasonic imaging catheter 20 in the heart 12 as shown in FIG. 2B allows a clinician to image the heart at the same time that detailed electrophysiology signals are obtained. Such simultaneous measurements can be diagnostically important especially when the heart suffers from arrhythmia, dyssynchrony, or other condition of irregular heart beat. In such cases, the electrophysiology catheter 64 records patterns of electrical activity flowing over the heart that may be causing the improper or mistimed contractions at the same time as the ultrasound imaging catheter 20 provides images of the chambers of the heart in motion.

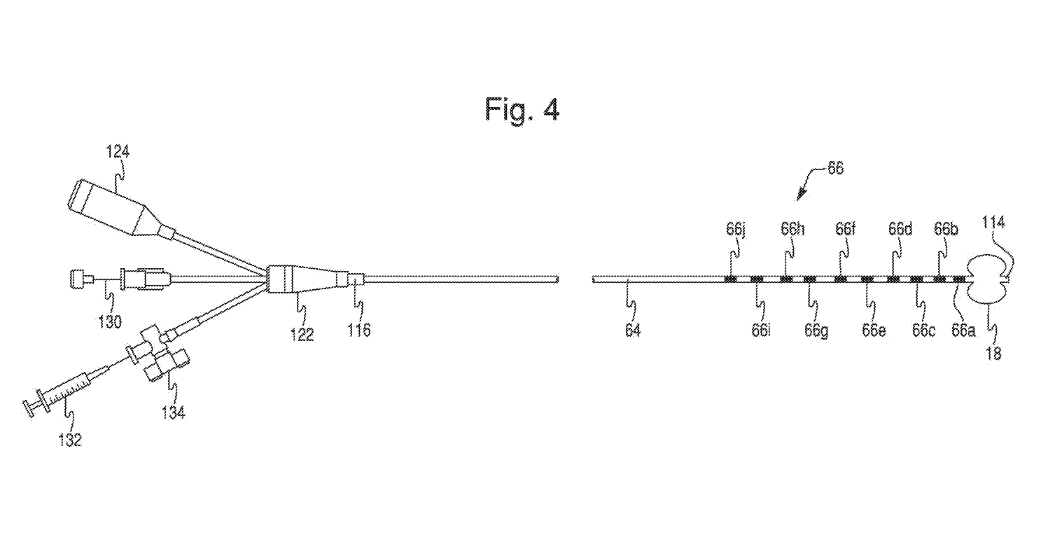

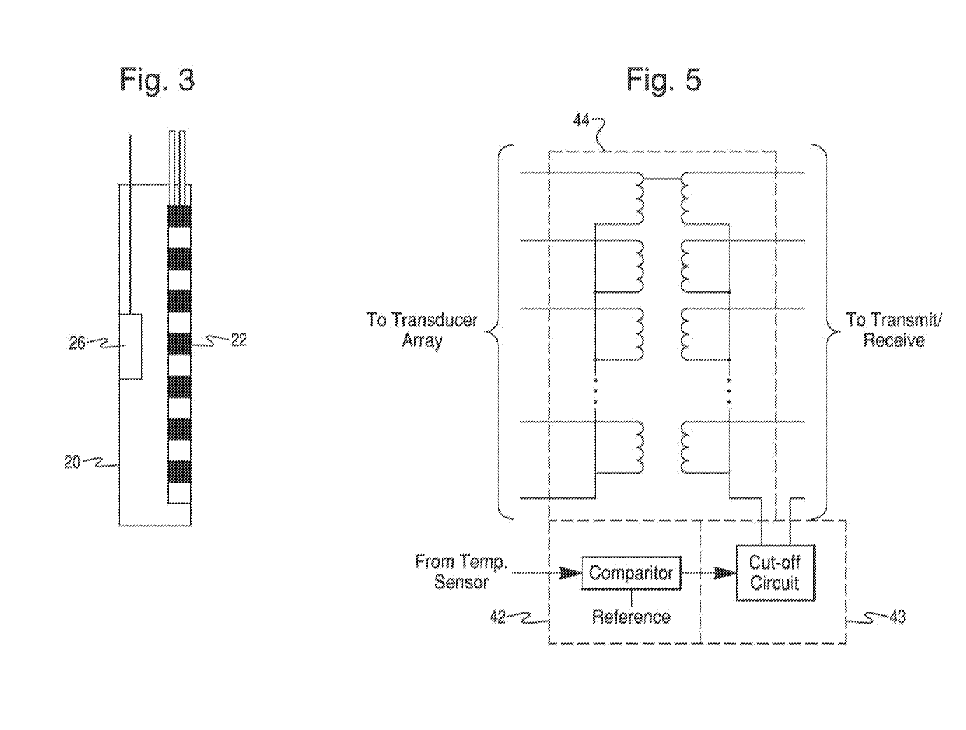

FIG. 3 is a close-up example of an embodiment of a portion of an ultrasonic imaging catheter 20, carrying an ultrasound transducer array 22. The array 22 may be located near the distal end of the catheter 20, but may be located elsewhere within the catheter 20.

There is a safety concern for intrabody ultrasound systems wherein the ultrasound power may locally heat tissue above a safe body temperature, particularly for the higher power employed by color Doppler imaging,. Although the ultrasound generation electronics may indirectly limit the amount of heat an ultrasonic catheter can theoretically induce in tissue at a given power level, direct monitoring of the actual temperature at the transducer array and surrounding tissue is much safer and avoids assumptions about how effectively specific tissues can dissipate the heat. Therefore, a need exists for a safety means either to warn the operator or to curtail the applied power automatically, whenever the measured temperature exceeds some pre-determined limit. An example of a standard safe temperature limit, as established by FDA in the United States, is 43 degrees Celsius, although the exact limit may depend on the specific environment and use of the catheter.

To address this safety concern, the catheter 20 may optionally further include an electronic temperature sensor 26, such as a thermocouple or thermistor, as shown in FIG. 3. The purpose of the temperature sensor 26 is to measure the increase in temperature resulting from the injection of high-power ultrasound into living tissue. Because the sound energy is most concentrated near the ultrasound transducer array 22, the temperature sensor 26 is best located very close to the array 22, such as on the catheter 20 containing the array 22. Temperatures above a proscribed level (e.g., 43° C.) can permanently damage tissue and must be avoided. Therefore, the temperature sensor 26, together with the thermal monitor circuit 42 of FIG. 5, can be calibrated to detect temperatures above the proscribed level. The temperature sensor 26 is connected to the thermal sensing circuit 42 and cut-off circuit 43 shown in FIGS. 1A-1E. When the temperature exceeds the proscribed temperature, the cut-off circuit 43 inhibits or at least reduces the generation of the ultrasound signals generated by the signal generator 46.

Examples of phased array ultrasound imaging catheters used in performing intracardiac echocardiography and methods of using such devices in cardiac diagnosis are disclosed in the following published U.S. patent applications—each of which is incorporated herein by reference in their entirety.
2004/0127798 to Dala-Krishna et al.;
2005/0228290 to Borovsky et al.; and
2005/0245822 to Dala-Krishna et al.
   Commercially available ultrasound catheters are available from EP MedSystems, Inc. of West Berlin, N.J.

It should be noted that the present invention is not limited to the specific ultrasonic imaging catheter assembly disclosed in the applications cited above, because the invention is applicable to various catheters and instruments designed for intravascular and intracardiac echocardiography and for other physiological uses involving an ultrasound beam former and interfaces with medical instruments and external display equipment.

FIG. 4 shows an embodiment of an electrophysiology catheter 64. Such catheters include a flexible elongated member with a distal end 114 and a proximal end 116, with an array of electrodes 66a-66j positioned on or near the distal end 114. A balloon 18 can be attached at the distal end 114 of the catheter 64. The catheter 64 may also include additional electrodes 67a

-67g (shown in FIG. 2B) located a distance away from the distal end 114 so that when the distal end 114 is positioned in the vicinity of the left atrium, for example, the additional electrodes 67a-67g are positioned in the vicinity of the right ventricle and/or right atrium, for example. The catheter 64 may also include other electrodes 65 (shown in FIG. 2B) located at other positions in order to sense electrical activity at other locations in the heart, such as low in the right ventricle 14 as shown in FIG. 2B. The flexible elongated portion of the catheter 64 may be made from extruded polyether block amide of the type sold by Atcochem North America, Inc. under the trademark PEBAX, but alternatively may be comprised of other polymeric materials with memory characteristics such as polyurethane, silicone rubber, and plasticized PVC etc.

Electrodes 66a-66j, 67a-67g may be spaced approximately 2 mm apart from each other on the catheter 64, with each electrode extending approximately 2 mm in length. Electrodes are preferably made of stainless steel, platinum, gold or other electrode material, and may be formed as thin flexible films applied to the exterior of the catheter body. The electrode array may extend over a length of approximately 35-40 mm of the catheter 64. Electrical wires (not shown) from each electrode are positioned within and pass through the interior of the catheter 64 to a manifold 122 secured to the proximal end 116 of the catheter 64. Each electrode can be coupled to its own connector 124 that can be connected to the electrophysiology equipment 60.

As illustrated in FIG. 4, the electrophysiology catheter 64 may also include additional ports which may be used, for example, to introduce a guide wire 130 into the catheter, to attach an inflation mechanism for inflating the balloon, or to attach a syringe 132 with a stopcock 134 which may be used to introduce various solutions into the catheter 64 during procedures.

An example of an electrophysiology catheter is the One-Piece™ Electrophysiology Catheter, part number EPC-E65P252, manufactured by EP MedSystems, Inc. of West Berlin, N.J.

FIG. 5 shows an embodiment of the isolation circuitry 44 and thermal monitor circuit 42. In this example embodiment, electrical isolation is accomplished by a transformer circuit for each transducer in the array 44. High frequency ultrasound signals (both transmitted and received) are communicated by means of isolation transformers within the isolation circuits 44, while direct and low frequency AC currents are electrically isolated. Transformers do not conduct direct current and small air-core transformers can be used that will not readily pass low frequency alternating current (such as the 50 to 60 hertz of standard utility power outlets). Thus, electrical isolation is provided by presenting high impedance to unintended direct current and to lower frequency alternating currents. Further, if insulated adequately, the transformers will not conduct D.C. voltages below some very high, specified break-down voltage.

FIG. 5 also illustrates an example embodiment of the thermal monitor circuit 42 that includes a thermal comparator circuit 42 coupled to a cut-off circuit 43. In this embodiment, signals received from a thermocouple or thermistor 26 positioned near the transducer array 22 can be compared in a comparator circuit to a reference threshold value corresponding to a maximum safe temperature. If the sensed temperature signal exceeds the reference threshold, indicating temperatures in the vicinity of the transducer array 22 exceed a safe temperature, the comparator circuit can generate a cut-off signal that is provided to the cut-off circuit 43. In the circuit embodiment illustrated in FIG. 5, a common lead is provided to all transformers, particularly on the transmit/receive side (i.e., the portion of isolation circuit connected to the ultrasound beamformer unit 40). This circuit permits the cut-off circuit 43 to be a simple switch that opens to disconnect the common lead on the transmit/receive side of the isolation circuit in response to a cut-off signal received from the temperature comparator circuit 42.

Another example embodiment of the thermal monitor circuit 42 includes a plurality of gate circuits configured to gate the individual leads passing signals to and from each of the transducer elements in the transducer array 22. So long as the temperature measured by the thermistor 26 remains below a safe level (e.g., not more than 43° C.), the gate circuits remain enabled allowing signals to pass to/from the transducer elements. However, should the temperature measured by the thermistor 26 reach or exceed an unsafe level, the thermal monitoring circuit 42 disables the gate circuits, automatically shutting off the transducer array 22. In another example embodiment, the thermal monitor circuit 42 is configured to disable transmission of ultrasound signals from the ultrasound beamformer unit 40 by disabling the transmit circuitry by signaling the signal generator 46 through a trigger mechanism, such as a hardware interrupt signal. Other thermal monitor circuit embodiments include circuits that disable an array of multiplexers or transmit channel amplifiers that may be used in the ultrasound beamformer unit 40 for generating, controlling, distributing, conditioning and/or transmitting ultrasound pulses to the transducer array 22. The various example embodiments of the thermal monitor and cut-off circuits 42, 43, as well as other suitable circuits, perform the safety function of discontinuing transmission of ultrasound signals from the signal generator 46 through the isolation circuit 44 upon receiving a cut-off signal or sensing an unsafe temperature in the vicinity of the transducer array 22.

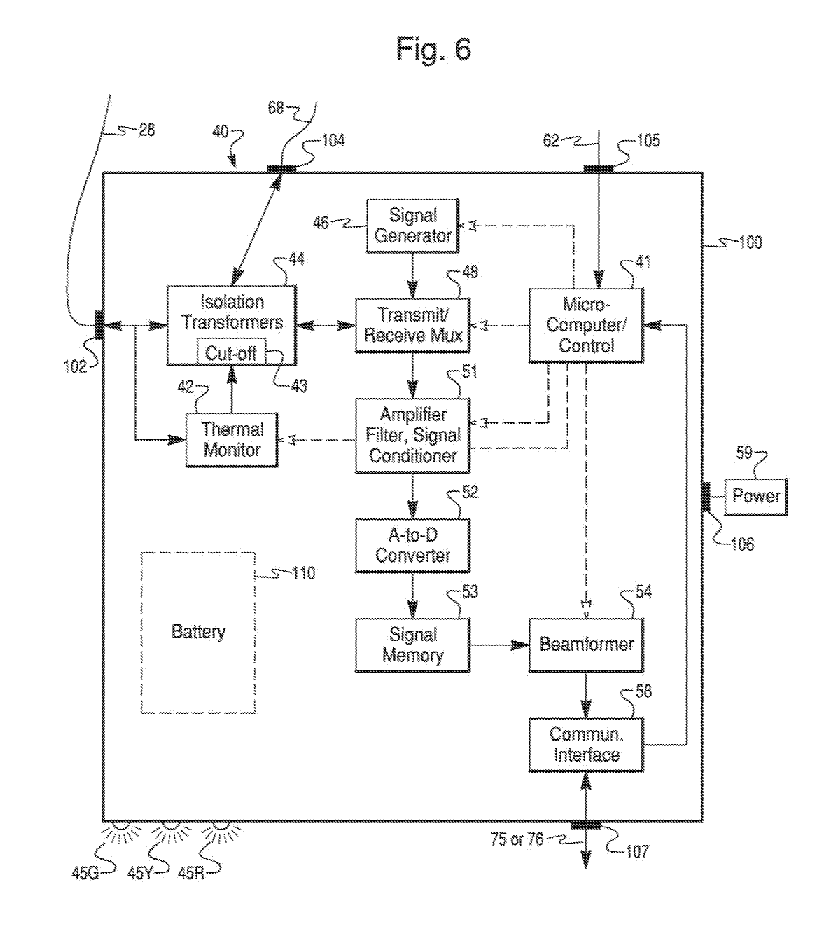

FIG. 6 illustrates example connections among components of the ultrasound beamformer unit 40. The embodiment illustrated in FIG. 6 is not intended to specify the only possible configuration of components and their interconnections but serves as an example of an enabling implementation.

The signal generator 46 generates electrical signals of ultrasonic frequencies, such as in the range of about 1 megahertz to 10 megahertz, as are commonly used for ultrasound imaging. The signals may be continuous or may be intermittent pulses. The electrical signals may pass through a transmit/receive multiplexer 48, isolation circuits 44, and a cable 28 to reach the transducer array 22. The electrical signals which reach the transducer array 22 cause the transducers to produce ultrasound signals in the same frequency range generated by the generator 46.

The transmit/receive multiplexer 48 directs the signals generated by the generator 46 through the isolation circuitry 44, which serves to limit unwanted electrical currents and voltages passing into the cable 28.

Signals which transit the isolation circuitry 44 pass into the signal cable 28 which delivers the signals to each of the transducers in the array 22. The cable 28 may include at least one wire per transducer in the array. The transducers in the array 22 convert the electrical signals into sound waves, which propagate into a portion of a patient's anatomy, such as the heart. As shown in FIG. 6, the same cable 28 (or a separate cable, not shown) may conduct the return ultrasound signals back to the isolation circuitry 44 of the ultrasound beamformer unit 40.

An embodiment of the isolation circuitry 44 is described above with respect to FIG. 5. In an embodiment of ultrasound beamformer unit 40, the isolation circuitry may be connected to the cable 28, with no other active circuitry in-between. This arrangement may prevent a possible, compromised path for leakage current caused by other circuitry. An embodiment employs each conductor of cable 28 both to conduct the generated electrical signal to each transducer of array 28 and to conduct the returning echo signal from the transducer. Thus, both the generated signal and the return signal may pass through the same isolation circuit (transformer, bi-directional optical isolator, or other electrical isolation component). In an embodiment there may be at least one isolation circuit for each transducer of array 28.

Multiplexer 48 may be necessary where the generated signals from the generator 46 and the received echo signals both pass through the same isolation circuitry 44 and the same wires of cable 28. Specifically, the transmit/receive multiplexer 48 may be used to separate out the received echo signals from the generated electrical signals from the generator 46. This may be accomplished by connecting the transducer array to the receiving amplifier circuits between generated ultrasound pulses, so the only signals allowed to pass are the received ultrasound echo signals. Alternatively, the electrical pulses of the transmitted pulses may be subtracted from all signals received from the isolation circuitry to yield the received ultrasound echo signals. The multiplexer 48 can direct the received ultrasound echo signals to conditioning circuitry 51 to filter and condition the signals as necessary. The signal can also be amplified, such as within the multiplexer 48, within the conditioning circuitry 51, or by a separate amplifier. At some point, the amplified and filtered echo signal can be digitized by an analog-to-digital converter (ADC) and can be temporarily stored in a signal memory buffer 53, such as random access semiconductor memory (RAM).

For an embodiment which employs separate transducers and conductors for the generated signals and for the received echo signals, separate transformers or mono-directional optical isolators may be used in the isolation circuitry 44. In such an embodiment, the transmit/receive multiplexer 48 may not be included.

The filter and conditioning circuitry 51 may be provided to reject signal frequencies outside a certain range, such as rejecting spurious signals induced in the cable 28, for example. The echo signals may be amplified as part of this circuitry (before, during, and/or after filtering), as part of the transmit/receive multiplexer 48 or elsewhere in the ultrasound beamformer unit 40. Other signal processing may be performed to enhance the desirable properties of the signal returned from the transducer array 22. For example, the signal conditioning circuitry 51 may reject the spurious signals from internal reflections within the catheter.

The analog-to-digital converter (ADC) 52 can be included to frequently sample the received ultrasound signals, which are received as analog electrical levels, and convert them to discrete digital numeric values. This conversion may be performed before, during, or following the action of the filter and conditioning circuitry 51. The signal may be in either analog or digital form or both during parts of the amplification, filtering, conditioning, and storage of the signal. In other words, the filter and conditioning circuitry 51 may apply well-known analog filtering techniques to the signals before digitization, may apply well-known digital filtering methods to the digitized signals, or do both. Techniques for such digital and analog signal processing are well known. There may be an ADC per transducer, or fewer ADCs may used with each being time-multiplexed among multiple transducers. Conversion of signals to streams of digital numbers is a convenience based on the current availability and economy of digital processing, but suitable analog circuits can be used in part or all of the ultrasound beamformer unit 40.

Because contemporary electronics routinely store signals in digital form, the embodiment of FIG. 6 may digitize the return signals prior to storage of some portion of the return signals in memory 53, which may be a semiconductor random-access memory (RAM). Methods of storing an analog signal, such as in time delay circuits, may be used instead of or in addition to digital storage. A portion of the memory 53 may store the signal from each transducer of array 22.

The beam-former 54 processes the returned echo signal data, some or all of which may be stored in memory 53. The beam-former 54 may calculate the amplitude of the ultrasound echo at each of many specific angles and distances from the transducer array. Techniques for beam-forming (either for transmission or for reception) are well known in the fields of ultrasound imaging and in phased array sonar and radar.

The result of the processing of the data stored in buffer memory 53 by the beam-former 54 typically can be a pixel-based image relative to a polar-coordinate system. The beam-former 54 may be implemented in very large scale integrated (VLSI) semiconductor circuits. The beam-former 54 may employ substantial parallel processing of the ultrasound signals from the transducers of array 22. For example, there may be simultaneous computations of more than one beam angle and/or more than one distance along a beam angle.

The amplitude, phase and time of arrival of reflected ultrasound pulses at each transducer in the array 22 can be used by the beam-former 54 to calculate the angle (with respect to the long axis of the transducer array 22) and distance from the array to each echo source. The distance and angle data may then be combined with amplitude (i.e., power of the received echo) to produce (e.g., by processing the data according to an algorithm) a pixel of image data. Alternatively, the beam-former 54 may store or output the processed received ultrasound as data sets comprising data groups of angle, distance and amplitude. In this and like manner, the beam-former 54 can turn the large volume of streaming ultrasound signals into a smaller set of data easily passed over a serial data link 75 for processing or display by a display unit 70. In an embodiment, the beam-former 54 is included in a VLSI chip within the ultrasound beamformer unit 40.

The beam former 54 may compare the frequency of the generated signals with the frequency spectrum of the returned echo signals. The difference in frequency relates directly to the velocity of tissue or blood toward (higher frequency) or away from (lower frequency) the transducer array due to the Doppler effect. The difference in frequency, i.e., the amount of Doppler frequency shift, is indicative of motion of the tissue (including blood) from which the ultrasound reflected. The frequency shift may be determined by mixing the generated signal and received echo signal and detecting the difference frequency. The conversion of the frequency shift to velocity depends on the speed of sound in the body, which is about 1450 to 1600 meters per second in soft tissue, including blood. The conversion of frequency shift to velocity according to well known algorithms may be performed immediately by the beam former 54, or later, such as by an external image processor at the time the image is displayed. If calculated by the beam-former 54, the velocity data (or Doppler shift) may be outputted as a fourth element of the data set, so that echo sources are identified by angle, distance, amplitude and velocity (or frequency or Doppler shift).

The velocity of echo sources may be computed at each of numerous angles and distances relative to the transducer array 22. The computed velocities may be represented visually as a spectrum of colors, for example, as is conventional in the art. The velocity of numerous points in the tissue or blood may be mapped to colors at the corresponding locations in the final image of the tissue. Image display unit 70, as shown in FIG. 1A, may perform this mapping, although it may be performed alternatively within ultrasound beamformer unit 40, as shown in FIG. 9.

With reference to FIG. 6, an embodiment of a beam former 54 may directly produce an image in rectangular coordinates. Alternatively beam former 54 may directly produce an image in polar coordinates and transform the image into rectangular coordinates. Alternatively, the beam former 54 may simply produce an image in polar coordinates (i.e., angle and distance coordinates) and allow subsequent image processing to perform the coordinate transformation as needed (such as in image display unit 70).

As also shown in FIG. 6, the buffer memory 53 may make available the return signal data representing the ultrasound echo waves, and the beam-former 54 may access that data and may calculate the amplitude of the ultrasound echo at each of many specific angles and distances from the transducer array. The result of the processing of the data stored in the buffer memory 53 by the beam-former 54 may be a pixel-based image relative to a polar-coordinate system. In an embodiment as illustrated in FIG. 6, polar-coordinate oriented ultrasound echo data may be serialized and may be transmitted to the image display unit 70 over data interface 75. Alternatively, the beam-former 54 may generate data relative to a rectangular coordinate system and transmit that data to display unit 70 over an interface 75.

A programmed microcontroller, microprocessor, or microcomputer 41 or functionally equivalent discrete electronics can be included to coordinate the activity described above within the ultrasound beamformer unit 40. In addition, the microcomputer 41 (or equivalent) may respond to configuration parameters and commands sent from the image display unit 70 (of FIG. 1A) over the communication interface 75 or 76 to the ultrasound beamformer unit 40.

In an embodiment, the ultrasound beamformer unit 40 may be configured via software or discrete circuits to adaptively cut and separate each frame of ultrasound image data. Such capability may be used to select and transmit frames for which there is useful information (e.g., changes in position of structures) to limit the bandwidth required for transmitting ultrasound images to external displays. In a normal cardiac cycle, portions of the heart are at rest for significant fractions of the cardiac cycle, so numerous images during such intra-contraction periods will contain the same image information. By not transmitting images in which there has been no change since the previous image, the same clinical information may be transmitted at substantially lower data rates. Such processing of image frames may be accomplished by a segmentation module (not shown).

In an embodiment associated with cardiac imaging, an external electrocardiogram (ECG) unit 60 (see FIG. 1A or 1B) may be connected to ultrasound beamformer unit 40 through an ECG communications interface 62. The signals sent through interface 62 may be used to synchronize the imaging with the heartbeat of the patient. For example, a sequence of images may be associated with a sequence of phases of a cardiac cycle, or images may be captured only at a specified phase of the cardiac cycle. The ECG communications interface 62 may be any wired or wireless interface. The ECG signal may be monitored by the microcomputer 41 to orchestrate the operation and timing of the signal generator 46 in order to image the heart at particular phases of the cardiac cycle.

Referring to FIGS. 1A and 1B, an embodiment may employ one or more ECG sensors 66 integrated in an intravenous catheter 64 that is coupled to the ultrasound beamformer unit 40 by a connecting cable 68. In an embodiment, ECG sensors may be included on the catheter 20 which carries the ultrasound transducer array 22. The ultrasound beamformer unit 40 may include connectors for receiving electrical connection plugs for the ECG catheter 64 or connecting the electrical connection plug on the cable 68. Such connectors may route the electrophysiology signals through the isolation circuitry 44 and then out to an external Electrophysiology analyzer 60 via cable 62. This embodiment allows the ultrasound beamformer unit 40 to serve as a universal connector for the ultrasound and electrophysiology catheters 64 used in a typical intracardiac examination employing both electrophysiology and ultrasound sensors. This embodiment reduces the need for multiple cables and connectors, thereby simplifying the procedure.

In an embodiment, signals from the electrophysiology catheter 64 may be used in lieu of, or in addition to, signals from the electrophysiology analyzer 60. The electrophysiology catheter 64 sensor signals can be used to record or control the timing of the ultrasound image acquisition relative to the cardiac cycle instead of or in conjunction with signals from the electrophysiology analyzer 60. The signals from an electrophysiology catheter 64 may be included within the data stream outputted by the ultrasound beamformer unit 40.

Whether an ECG signal is acquired from an external Electrophysiology analyzer 60 or an attached ECG sensor 66, the interface 60, 68 or cable 28, respectively, may be electrically isolated from the ultrasound beamformer unit 40 to enhance patient safety and reduce electronic noise in the system. For wired interfaces, the isolation may be accomplished by a transformer isolation circuit 44 or an optical isolator as described herein. Protection against electrical leakage currents and high voltage discharges may be accomplished in an embodiment by using a wireless interface for the ECG interface 62.

In addition to including connectors for receiving the input/output connection plugs for ultrasound catheters and ECG sensors or equipment, some embodiments of the ultrasound beamformer unit 40 include connections for additional sensors, such as intracardiac percutaneous leads, subcutaneous leads, reference leads and other electrical leads that may be employed during a procedure. As with ultrasound and ECG connections, such additional lead connections can be coupled to isolation circuitry 44 to provide patient protection. By providing such connections with integrated isolation circuitry, the ultrasound beamformer unit 40 can serve as a central interface unit for connecting all sensors employed in a procedure.

Some or all of the electronic circuitry of ultrasound beamformer unit 40 may be implemented within one or more large scale integrated circuits such as VLSI, ASIC, or FPGA semiconductor chips, as are well known in the electronics art. The program instructions running in the microcomputer 41 may be stored in some form of random access memory (RAM) or read-only memory (ROM) as software or firmware.

Portable ultrasound units, which contain compact signal generation and beam-forming circuitry and which are separate from the display unit 70, are available commercially from Terason, a division of Teratech Corporation (Burlington, Mass.). Some of the details of these portable ultrasound units are described along with associated methods in the following published patent applications—each of which is incorporated herein by reference in its entirety:
2005/0228276 to He et al;
2005/0018540 to Gilbert et al; and
2003/0100833 to He et al.
   Also described in the above applications are methods of implementing the beam-forming computations.

As shown in FIG. 7, the communication interface 74 within the display unit 70 may receive the ultrasound data over the interface 75 or 76 and may temporarily store the data in memory 77 for further processing. The image data at this point may be relative to a polar coordinate system, so scan converter 82 may reformat it into an image relative to a rectangular coordinate system as needed.

Image data from the scan conversion (and Doppler processing, if any) may be processed by an image renderer 83, formatted, and displayed as an image on a video monitor 73. For example, the rendering circuit 83 may generate a gray-scale image (such as a B-mode image) in which the brightness of each pixel is representative of the amplitude of the ultrasound echo from the anatomical point to which the pixel corresponds.

Besides responding to operator input of configuration information, the interactive control 80 may also respond to operator input controlling how the image is converted, processed, and rendered on the display 73.

The image display unit 70 may perform other functions. For example, the interactive control 80 in the image display unit 70 may transmit configuration parameters and control commands to the ultrasound beamformer unit 40, where the configuration parameters and commands may be supplied by the operator by means of interactive inputs from a pointing device (mouse, trackball, finger pad, or joystick, for example) and a keypad or keyboard 72 attached to display unit 70.

Optionally, the interactive control 80 of the image display unit 70 may forward the image and/or raw data to a network file or database server, to the Internet, to display screen, or to a workstation through a communication interface 92.

FIG. 8 illustrates example images that can be displayed by an embodiment. Contained within the field of view 8 of the B-mode image is an image of the walls 5 of a ventricular cavity 4. Also shown in FIG. 8 is a plot 9 of the blood flow velocity as derived from the spectral analysis of the Doppler frequency shifts.

In an embodiment, the image display unit 70 circuitry may be included within the ultrasound beamformer unit 40 housing or chassis. This may be accomplished by simply including the image display unit 70 circuitry as another board or VLSI chip within the ultrasound beamformer unit 40. Alternatively, the circuitry and functionality of the components of the image display unit 70 may be incorporated in a VLSI chip that also encompasses the beam-former 54 and/or microcomputer 41 within the ultrasound beamformer unit 40. In such an embodiment, the ultrasound beamformer unit 40 outputs image data as a video signal (e.g., VGA, composite video, conventional television or high-definition video) that can be carried by a cable 75 directly to a display 73 to yield an image on the screen without further processing. In a further embodiment, the ultrasound beamformer unit 40 may output image data as a network compatible signal, such as Ethernet or WiFi, that can be directly coupled to a network.

FIG. 9 is a block diagram of such an embodiment in which most or all of the image processing circuitry is included within the ultrasound beamformer unit 40. That is, most or all of the circuitry of FIG. 7 is compactly incorporated into the chassis of ultrasound beamformer unit 40. This is enabled by readily available circuitry equivalent to a personal computer on a small single circuit board or within a single integrated circuit.

In the embodiment illustrated in FIG. 9, the communication interface 58, the communication cable 75 (of FIG. 6) and the communication interface 74 (of FIG. 7) may be simplified or essentially eliminated. Further, the ultrasound beamformer unit 40 may directly link to the hospital network and infrastructure 90 (shown in FIG. 1) through wired or wireless link 92 as described above.

A user input device 72 may connected to the ultrasound beamformer unit 40 to permit a user to provide commands and operating parameters, such as by way of a keyboard, keypad and/or a user pointing device such as a mouse, touch screen, trackball, light pen, or finger pad. The user input device 72 may include a voice recognition device in lieu of or in addition to a keyboard. The input device 72 may be connected to the ultrasound beamformer unit 40 by a cable, an infrared link, a radio link (such as Bluetooth), or the equivalent, each of which are commercially available.

A display monitor 73 may not be present as part of ultrasound beamformer unit 40. Any of many choices, sizes, and styles of a display 73 may be connected to ultrasound beamformer unit 40. For example, the external display monitor 73 may be a cathode ray tube, a liquid crystal display, a plasma display screen, “heads up” video goggles, a video projector, or any other graphical display device that may become available. The display monitor 73 may be large and may be located conveniently out of the way, such as a plasma screen hung from the ceiling or on a wall. The display monitor 73 may be positioned for better viewing by the physician. There may be more than one display 73, and a display may be positioned remotely, such as in another room or in a distant facility. The display 73 may be connected to the ultrasound beamformer unit 40 by a cable, an infrared link, a radio link (such as Bluetooth), or any equivalent wireless technology.

In an embodiment, the display monitor 73 and/or the user input device 72 may be embodied by a computer terminal, workstation, or personal computer such as a laptop computer. Such an embodiment can be configured to display the graphical output from the image rendering circuits 83 and to pass user inputs on to the interactive control 80 of the ultrasound beamformer unit 40. Alternatively, in an embodiment in which the display monitor 73 and user input device 72 are provided by a computer system, the computer system may operate software enabling it to perform additional image processing on the data received from the ultrasound beamformer unit 40.

The ultrasound beamformer unit 40 may need to be powered by an external power source 59, as was previously discussed with respect to FIG. 1A and 1B. The power source 59 of FIG. 9 may be a separate external power supply with provision to isolate the ultrasound beamformer unit 40 from the patient. The power source 59 may be a power source, such as batteries, contained within the ultrasound beamformer unit 40. Alternatively, the power source 59 may simply be conductors in a data cable supplying power from input unit 72 or display unit 73.

Because of the priority of safety for a patient, a wired connection between the ultrasound beamformer unit 40 and the user input device 72 may need isolation circuitry to prevent potentially harmful leakage current from flowing from the input device 72 through the ultrasound beamformer unit 40 to the patient. This isolation may be provided by the isolation circuits 44 within the ultrasound beamformer unit 40 shown in FIGS. 1A, 1B, 4, 6 and 8. Alternatively or in addition, isolation circuits may be provided between the ultrasound beamformer unit 40 and the user input device 72, such as an optical fiber data link or optical isolation module as previously described. For example, if the input device 72 is powered from a source which shares a common ground (or other low impedance path) with the patient, there could be unexpected potential differences between the input device 72 and the patient conducted through the ultrasound beamformer unit 40. Such a potential difference could produce harmful leakage currents. Such currents are limited by sufficiently high impedance, such as provided by isolation circuitry, such as an optical isolator. The isolator may also protect against unintended high voltages caused by failures in the input device 72, its power source, or by lightening, as well as reduce electronic noise induced by stray electromagnetic radiation. The same reasoning applies to a wired connection between the display 73 and the ultrasound beamformer unit 40, and any connection to a network.

One way to achieve isolation for the input device 72 or display device 73 is to employ a wireless communication link between device 72 and unit 40 and between device 73 and unit 40. Any wireless communication link of sufficient bandwidth, such as those mentioned previously, may be used in this capacity.

The embodiment illustrated by FIG. 9 includes an optional connection 68 between the ultrasound unit and an ECG sensor, such as an ECG catheter 64, and an optional connection 62 between the ultrasound beamformer unit 40 and an external Electrophysiology analyzer 60. In addition or alternatively, an embodiment may employ an ECG sensor integrated in a catheter 20 with a connection to ultrasound beamformer unit 40 through cable 28. The foregoing comments in the discussion of FIG. 6 about ECG signals also apply to an embodiment as illustrated by FIG. 9. Interface 62 may be any wired or wireless communication interface discussed previously.

As illustrated in FIGS. 5 and 8, various embodiments can be housed within a housing 100 providing environmental and electrical isolation for the circuitry (e.g., circuit boards and integrated circuits) of the ultrasound beamformer unit 40. This housing 100 can be fabricated from nonconductive material, such as plastics, to minimize stray currents due to induction. The housing may also be fabricated from materials that can withstand high temperatures and/or gamma radiation so that the housing can be sterilized by heat or exposure to gamma rays.

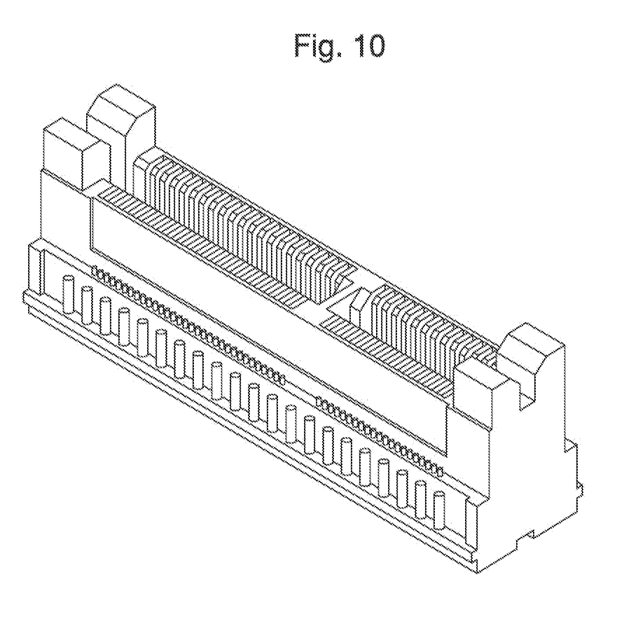

Referring to FIGS. 6 and 9, the housing 100 can include electrical connectors to permit the ultrasound beamformer unit 40 to be quickly connected to sensors, power, a user interface and displays. For example, one or more multi-contact ultrasound catheter connectors 102 may be provided to enable a reliable electrical connection between an ultrasound phased array catheter 20 cable 28 plug and the ultrasound beamformer unit 40 while maintaining an environmental seal. An example of a suitable ultrasound catheter connector 102 is a card edge connector, such as the 0.762 mm Pitch Hi-SpecGS™ Memory Expansion Card Edge Connector manufactured by Molex Corporation (www.molex.com) illustrated in FIG. 10. Such an ultrasound catheter connector 102 can be provided on the interior of the housing 100. The ultrasound catheter connector 102 can be directly connected to the isolation circuits 44, with an electrical lead from a temperature sensor 26 connected to the thermal monitor circuit 42. A card connector (which is a flat plug) on the cable 28 can be used to pass through a sterile plastic barrier to establish an electrical connection with the connector 102. In this manner, a sterile plastic barrier can be used to serve as a boundary between sterile and non-sterile environments, and may be disposable to allow re-use of one or more of the various components. As described above, the plastic barrier may comprise, for example, a plastic sleeve or bag that encloses the housing 100 so the ultrasound beamformer unit 40 can be positioned near the patient and within a sterile boundary.

Additionally, one or more ECG sensor electrical connectors 104 may be provided in the housing 100 to permit electrically connecting ECG sensor connectors 68 to the ultrasound beamformer unit 40 without compromising the environmental seal of the housing 100. On the inside of the housing 100, the ECG sensor electrical connectors 104 may be electrically coupled to the isolation circuit 44 or passed through to an external Electrophysiology analyzer 60 via an ECG unit connector 105. In an embodiment, the housing 100 includes sixty-four or more contact edge connector type ports 105 for connecting sixty-four individual ECG probe elements.

Similarly, a power connector 106 may be provided in the housing 100 for accepting a connector to an external power source 59. Alternatively or in addition, an internal power source, such as an optional battery 110 can be included in the housing 100. Additionally, an output connector 107 may be provided for connecting to an external computer or display unit 73. For example, the output connector 107 may be a video connector (e.g., CGI, VGA or composite video) or a standard two-direction serial connector such as USB, FireWire or fiber optic connector. Similarly, an input connector 108 may be provided in the housing 100 to connect to user interface devices, such as a keyboard or computer 72. Likewise, a network cable connector 109 may be provided for directly connecting the ultrasound beamformer unit 40 to a network.

Full system implementations of various embodiments are illustrated in FIGS. 11 and 12. In the embodiment illustrated in FIG. 11, the electrophysiology analyzer 60 is packaged in a single workstation 69 which is connected by a data and power cable 75/62 to the ultrasound beamformer unit 40. Ultrasound imaging catheter(s) 20 and electrophysiology catheter(s) 64 connect to the ultrasound beamformer unit 40 by cables 28 and 68, respectively, as described herein. The workstation 69 is connected to display(s) 73 and user input devices, such as a keyboard 72a and a mouse 72b. The workstation 69 includes computer processors operating program software instructions which cause the workstation to perform the functions of electrophysiology analysis, as well as control of ultrasound imaging equipment, including the ultrasound beamformer unit 40, and the processing of ultrasound images obtained from an ultrasound imaging catheter 20. Such software is stored on machine readable media, including random access memory (RAM) within the processor, optionally read-only memory (ROM), hard disc storage, and compact disc storage.

Functionality provided in software instructions stored on computer readable memory and implemented on the workstation 69 processor include: receiving, storing and analyzing electrophysiology signals received from an electrophysiology catheter; generating displays of electrophysiology data for presentation on a display 73; generating menu screens for presentation on a display 73 to control electrophysiology data analysis, storage and display; receiving and processing user input in response to electrophysiology menu screens; generating menu screens for presentation on a display 73 to control ultrasound image equipment (including control of the beamformer unit 40); generating menu screens for presentation on a display 73 to control ultrasound image signal data analysis, storage and display; receiving and processing user input in response to ultrasound imaging and analysis menu screens; generating and transmitting control commands to the ultrasound beamformer unit 40 to control operation of the ultrasound imaging equipment; receiving, storing and analyzing ultrasound image data received from the ultrasound beamformer unit 40; generating displays of ultrasound image data for presentation on a display 73; and generating combined displays of electrophysiology data and ultrasound images for presentation on a display 73. The software instructions may further enable the workstation 69 to receive, process and display images and data from other sensors, including for example, X-ray images, such as X-ray images of the heart that can image the catheters at the same time that electrophysiology and ultrasound image data is obtained. The software instructions implemented on the workstation 69 configure the workstation 69 to provide commands to the ultrasound beamformer unit 40 that may include but not be limited to:

Record image;

Freeze image;

Switch modes;

Capture;

Brightness;

Contrast;

Ultrasound Power;

Frequency;

Pulse repetition rate (PRF);

Color area (for color Doppler mode);

Scale;

Focus zone; and

Scan angle.

The software instructions also configure the workstation 69 to receive data and status signals from the ultrasound beamformer unit 40. These data signals and configuration data may include but not be limited to: radiofrequency signals (echo data information); vector processing; scan conversion; and display.

Integrating the ultrasound system control and processing with the electrophysiology system also enables the ultrasound control and data recording to be coordinated with ablation therapies which are controlled by some electrophysiology systems. In an ablation operation, high power radiofrequency energy is applied to tissue through an electrode on a catheter in order to kill a region of heart tissue. Some electrophysiology catheters include one or more ablation electrodes so the same catheter can be used to receive electrophysiology signals and conduct ablation. When ablation RF power is applied, the resulting signal can overwhelm the electrophysiology system, so normally, electrophysiology recording is halted during the time when ablation is applied to the heart. However, ultrasound imaging (and recording of ultrasound images) can proceed during ablation. Thus, an integrated ultrasound/electrophysiology system can control and coordinate ultrasound imaging to ablation therapy, such as by recording ultrasound images during ablation, as well as to electrophysiology monitoring.

FIG. 12 illustrates a typical implementation of various embodiments. Typical implementations include the ultrasound beamformer unit 40, workstation processor 69, displays 73a-73c, user input devices 72a, 72b, ultrasound imaging catheter 20 and electrophysiology catheters 64 illustrated in FIG. 11. Additionally, a typical implementation may include multiple displays, such as a menu/control display 73a, and one or two data displays 73b, 73c. Multiple displays allows a clinician to view all of the data being gathered, raw data and processed information displays, current data and historic data, and other combinations of current and stored data as may facilitate diagnosis. For example, FIG. 12 illustrates an operating configuration in which system control menus and configurations are presented on display 73a, a combined display of ultrasound images, X-ray image and electrophysiology is presented on display 73b, and electrophysiology data alone are presented on display 73c. A typical implementation may be integrated into/onto a rollable desk 160 or cart to enable the system to be repositioned within the catheter laboratory and moved close to a patient. The system may also include a printer 163, a stimulator 161 for stimulating heart tissue during electrophysiology examination, and an amplifier 162 for amplifying electrophysiology signals before connection to the workstation processor 69. The amplifier 162 may include amplifier circuits, filter circuits to reject noise (such as 60 Hz noise induced from electrical equipment in the vicinity) and analog-to-digital converter circuits, the combination of which are referred to herein as electrophysiology signal processing circuitry. Also shown in FIG. 12 is electrophysiology catheter connector box 164 which provides several connect electrical connectors for connecting leads from the electrophysiology catheters 64. The connector box 164 then connects to the workstation processor 69 by a connection cable 62. The connector box 164 may be connected by the cable 62 to the amplifier 162.

An advantage of the embodiments described above is the ability to gather, correlate and co-display electrophysiology data and intracardiac ultrasound images simultaneously. FIG. 13 shows an example display screen 170 which includes a presentation of electrophysiology data 171 positioned side-by-side with live ultrasound images 172. So presented, the clinician can observe both the electrical signals passing through heart tissue and the movements of the heart tissue in response. Such a display may also include a summary of system parameters, settings and patient vital signs in a data window 173, as well as ultrasound operational or control parameters in a control window 174. As a further example, FIG. 14 shows a display screen 170 which includes a presentation of electrophysiology data 171 positioned side-by-side with live ultrasound images 172 and X-ray images 175.

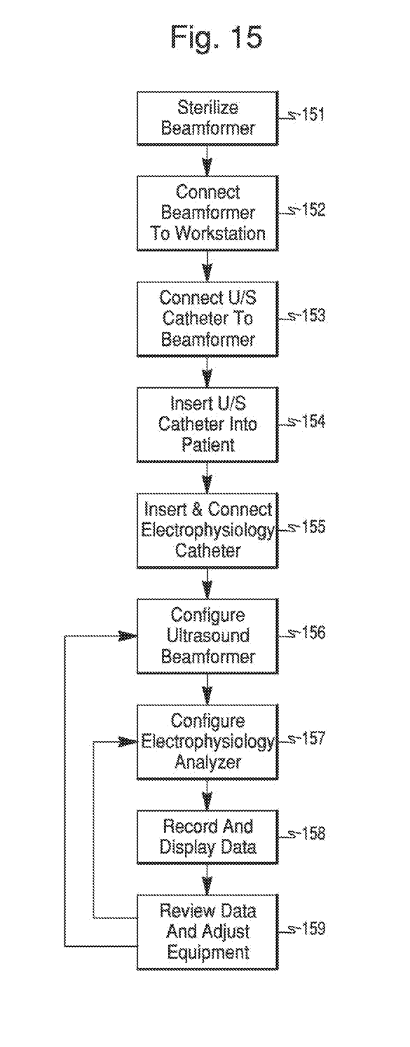

The various embodiments may be used according to the following method which is illustrated in FIG. 15, wherein the steps can be performed in an order other than that described below. At least some of the steps may be performed contemporaneously. Some of the steps are optional.

An ultrasound beamformer unit 40, such as shown in FIG. 9, may be sterilized or placed inside an externally sterile enclosure, step 151. The ultrasound beamformer unit 40 may be positioned next to a patient, such as on the examination table on which the patent lies. The ultrasound beamformer unit 40 may be connected to an external power source or an internal power source.

The ultrasound beamformer unit 40 then connected to the combined workstation 69, step 152. The workstation 69 is connected to a display monitor 73 may be supplied and connected to the ultrasound beamformer unit 40. A data interface 92 may also be established between the workstation 69 and a clinic or hospital infrastructure 90.

A sterile ultrasound transducer cable 28 is connected between the ultrasound beamformer unit 40 and an ultrasound imaging catheter 20, step 153. The ultrasound catheter 20 is introduced into the patient's body, such as by percutaneous cannulation, and positioned so the transducer array 28 is at a desired location and orientation, such as guided by use of fluoroscopy, step 154. The transducer array 28 may be dynamically repositioned to other locations and orientations.

One or more sterile electrophysiology catheters 64 is introduced into the patient's body, such as by percutaneous cannulation, and positioned so the electrodes are at desired locations in the heart, step 155. Positioning of the electrophysiology catheters 64 may be facilitated with fluoroscopy and/or ultrasound imaging using the ultrasound imaging catheter 20. Once positioned in the desired location, the electrophysiology catheters 64 are connected to isolation circuits in the ultrasound beamformer unit 40 or separate isolation box.

Using menu screens presented on the integrated workstation 69 and user input devices 72, a clinician can initialize and configure the ultrasound beamformer unit 40, step 156. The configuration step may include setting of operational parameters of the ultrasound beamformer unit 40, such as ultrasound frequency, mode of operation, mode of image processing, characteristics of the transducer array 22, anatomical position of the array 22, details about the patient, and so forth. The operating parameters may be changed during operation of the invention. For example, the frequency may be changed to increase or decrease the depth of penetration of the ultrasound energy. As another example, the mode of display may be changed from B-mode (which may help the operator position the array 22 with respect to anatomy) to Doppler mode (e.g., color Doppler) to observe and measure the velocity of motion of blood.

Using menu screens presented on the integrated workstation 69 and user input devices 72, a clinician can initialize and configure the electrophysiology analyzer 60 portion of the workstation 69 to receive, record and display electrophysiology signals received from the electrophysiology catheter(s) 64, step 157.

Once configured, the clinician may then record and display electrophysiology data and ultrasound images, step 158. Data may be stored on hard disc storage and/or transmitted to other locations, such as over the internet. The clinician can review the data on displays and, based on the data obtained, make adjustments to the location of catheters and/or settings of the electrophysiology analyzer 60 or ultrasound beamformer unit 40 settings, step 159.

An embodiment includes some or all of the components described herein as a packaged kit with previously sterilized contents. The contents may include, by way of non-limiting example, a catheter 20 bearing an ultrasound transducer array 22, one or more sterile cables, a sterile enclosure for the ultrasound beamformer unit 40, a battery for the ultrasound beamformer unit 40 (if it is battery powered), and instructions. The kit may further include a cable to connect the ultrasound transducer array 22 to the ultrasound beamformer unit 40. The kit may include a cable to connect the ultrasound beamformer unit 40 to the display unit 70, unless the connection between them is wireless. In lieu of just a sterile enclosure for ultrasound beamformer unit 40, an embodiment of the kit may contain the ultrasound beamformer unit 40 which has been previously sterilized. Any appropriate method may be used to sterilize the contents, such as gamma radiation, which is typically used to sterilize packaged kits in bulk. Some or all of the contents may be disposable or may be sterilizable for reuse.

While the present invention has been disclosed with reference to certain exemplary embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

What is claimed is:

1. An apparatus for connecting multiple catheters at the same time, the apparatus comprising:
a housing;
a first port positioned on the housing and configured to electrically couple with an ultrasound catheter; and
a second port positioned on the housing and configured to electrically couple with an electrophysiology catheter.

2. The apparatus of claim 1, further comprising a third port positioned on the housing, wherein the third port is electrically coupled to first port.

3. The apparatus of claim 2, wherein the third port comprises a high density connector.

4. The apparatus of claim 2, wherein the third port is electrically coupled to the second port.

5. The apparatus of claim 2, further comprising a fourth port positioned on the housing and configured to couple with a positioning catheter.

6. The apparatus of claim 1, wherein the housing is sized and configured to attach to a bed of a patient.

7. The apparatus of claim 1, further comprising an isolation circuit electrically coupled to one of the first port and the second port.

8. The apparatus of claim 1, further comprising a current isolator coupled to the first port and to the second port, wherein the current isolator is configured to limit an amount of current passing to the first port and to the second port.

9. The apparatus of claim 8, wherein the current isolator comprises a first isolation circuit electrically coupled to the first port and a second isolation circuit electrically coupled to the second port.

10. The apparatus of claim 9, further comprising a cable electrically coupled to the first isolation circuit, wherein the cable is configured to couple with ultrasound equipment.

11. The apparatus of claim 8, wherein the current isolator is configured to allow the ultrasound catheter and the electrophysiology catheter to operate at the same time.

12. The apparatus of claim 8, wherein the current isolator is positioned within the housing.

13. The apparatus of claim 1, further comprising the ultrasound catheter coupled to the first port.

14. The apparatus of claim 13, further comprising the electrophysiology catheter coupled to the second port.

15. An apparatus for electrically isolating catheters, the apparatus comprising:
a housing;
a first isolation circuit positioned within the housing, the first isolation circuit comprising a first set of transformers that are configured to be electrically coupled to a first catheter; and
a second isolation circuit positioned within the housing, the second isolation circuit comprising a second set of transformers that are configured to be electrically coupled to a second catheter;
wherein the first catheter is of a different type than the second catheter, and wherein the first isolation circuit and the second isolation circuit are configured to allow the first catheter and the second catheter to operate at the same time.

16. The apparatus of claim 15, wherein the first catheter comprises an ultrasound catheter and wherein the second catheter comprises an electrophysiology catheter.

17. The apparatus of claim 15, wherein the first isolation circuit is configured to be electrically coupled with ultrasound equipment for controlling an ultrasound transducer assembly located in the first catheter.

18. An interface for limiting an amount of current passing to an intra-body medical device, the interface comprising:
a housing;
a first catheter port positioned on the housing and configured to couple with a first catheter;
a first processor port positioned on the housing and configured to couple with a first cable linkable to a first processor;
a second catheter port positioned on the housing and configured to couple with a second catheter, wherein the second catheter is of a different type than the first catheter;
a second processor port positioned on the housing and configured to couple with a second cable linkable to a second processor; and
a current isolator coupling the first catheter port to the first processor port and the second catheter port to the second processor port, wherein the current isolator is configured to limit an amount of current passing to the first catheter port and to the second catheter port.

19. The interface of claim 18, wherein the first catheter comprises an ultrasound catheter and wherein the second catheter comprises an electrophysiology catheter.

20. The interface of claim 18, wherein the current isolator is configured to limit the amount of current passing to one of the first catheter port and the second catheter port to not more than about 50 μA.
